(19) United States
(12) Patent Application Publication
(10) Pub. No.: US 2004/0228794 A1
(43) Pub. Date: Nov. 18, 2004

(54) Therapeutic agent carrier compositions

(75) Inventors: Richard E. Weller, Selah, WA (US); Darrell R. Fisher, Richland, WA (US); Michael A. Lind, Kent, WA (US); Allison A. Campbell, Kennewick, WA (US); Anna Gutowska, Richland, WA (US); Craig F. Habeger, Chillicothe, IL (US)

Correspondence Address: KLARQUIST SPARKMAN, LLP, 121 SW SALMON STREET, SUITE 1600, ONE WORLD TRADE CENTER, PORTLAND, OR, 97204, US

(73) Assignee: Battelle Memorial Institute

(21) Appl. No.: 10/784,147

(22) Filed: Feb. 19, 2004

Related U.S. Application Data
(63) Continuation-in-part of application No. 09/994,509, filed on Nov. 26, 2001, now abandoned.
Application No. 09/994,509 is a continuation-in-part of application No. 09/853,507, filed on May 10, 2001.
Application No. 09/853,507 is a continuation of application No. 09/058,712, filed on Apr. 10, 1998, now Pat. No. 6,296,831.
(63) Continuation-in-part of application No. 10/124,614, filed on Apr. 16, 2002.
Application No. 10/124,614 is a continuation-in-part of application No. 09/833,460, filed on Apr. 11, 2001.
(60) Provisional application No. 60/236,926, filed on Sep. 28, 2000.

(51) Int. Cl. A61K 51/00; A61K 33/42

(52) U.S. Cl. 424/1.11; 424/604

(57) ABSTRACT

Disclosed herein are therapeutic agent carrier compositions that include a carrier comprised of a stimulus sensitive gel, fibrin, or combinations thereof, and a therapeutic agent such as an aqueous insoluble or confined radioisotope. Stimulus sensitive gels include thermally reversible stimulus-sensitive gels that are linear random copolymers of a [meth-]acrylamide derivative and a hydrophilic comonomer; thermogelling biodegradable polymers, which include biodegradable polymers including a linked polyethylene glycol (PEG) block and biodegradable polyester block; and enzymatically degradable polypeptide poylmers. In certain embodiments the therapeutic agent is a hydrothermally synthesized radioactive therapeutic agent, such a 90-yttrium phosphate colloid. Methods of making such carrier compositions and methods of using such carrier compositions to treat diseases such as cancer also are disclosed. For example, the carrier compositions can be injected into a tumor to treat a cancer and can be applied to the margins of a resected tumor to destroy vestigal cancer cells.

### CROSS REFERENCE TO RELATED APPLICATION

This disclosure is a Continuation-In-Part of application Ser. No. 09/994,509, filed Nov. 26, 2001, which is a Continuation-In-Part Application of application Ser. No. 09/853,507, filed May 9, 2001, which is a Continuation of application Ser. No. 09/058,712, filed Apr. 10, 1998, now U.S. Pat. No. 6,296,831, and is a Continuation-in-Part of application Ser. No. 10/124,614, filed Apr. 16, 2002, which is a Continuation-in-Part of application Ser. No. 09/833,460, filed Apr. 11, 2001, which application claims the benefit under 35 U.S.C. §119(e) of provisional Patent Application No. 60/236,926, filed Sep. 28, 2000, which are all incorporated by reference herein.

The disclosed compositions were made with Government support under Contract DE-AC06 76RLO 1830 awarded by the U.S. Department of Energy. The Government has certain rights in this patent.

### FIELD

The disclosed gels and matrices are used as carriers for the disclosed therapeutic agents in the treatment of disease.

### BACKGROUND

Radiolabelling as a method of diagnosis or treatment has been in use for many years. The continuing challenge has been to maximize concentration of the radioisotope in the area or region of interest, diseased tissue or tumor, while minimizing the concentration of the radioisotope in other areas and thereby minimizing damage to healthy tissues.

The paper by S. Ning, K. Trisler, D. M. Brown, N. Y. Yu, S. Kanekal, M. J. Lundsten, S. J. Knox: “Intratumoral radioimmunotherapy of a human colon cancer xenograft using a sustained-release gel,” 39, Radiotherapy and Oncology, 179-189, 1996 discusses an intratumoral injectable gel drug delivery system for local administration of radio-immunotherapy. The injectable gel was a collagen-based drug delivery system designed for intratumoral administration. The study demonstrated that intratumoral delivery of radiolabeled antibodies using the collagen gel system markedly increased the retention of radioisotope in the tumors, enhanced the antitumor efficacy, and reduced the systemic toxicity compared to systemic administration of the radiolabeled antibody. Ning et al. teach the use of injectible collagen gels that are not stimuli-sensitive. Moreover, these collagen gels neither fully perfuse tumor tissue nor do they hold the radioisotope within the collagen gel matrix. Thus, the radioisotope is attached to an antibody for perfusing and binding to the tumor tissue. Lack of perfusion of the collagen gel and limited range of radioisotope decay products require that the radioisotope leave the collagen gel matrix to achieve close proximity with tumor tissue to achieve the therapeutic effect.

In the paper Preliminary Experience of Infusional Brachytherapy Using Colloidal ³²P, S E Order, J A Siegel, R Principato, L S Zieger, E Johnson, P Lang, R Lustig C Kroprowski, P E Wallner, 25 Annals Academy of Medicine (1996),an infusion by a needle into a tumor was done without the need for an arterial catheter and eliminating the need for hospitalization. This paper reports using dexamethasone (Decadron) to overcome intratumoral resistance followed by macroaggregated albumin then colloidal chromic phosphate ³²P followed by more macroaggregated albumin injected into the tumor. Sufficient radiation emitted by the radioisotope leads to tumor cell killing and remission of solid cancers. However, disadvantages of this method include the serial injections and leakage of ³²P from the tumor.

There is need in the art for a method of introducing a radioisotope into a localized area with a single or multiple injection(s) as well as a need for a local delivery system with little or reduced leakage of the radioisotope.

Stimulus-sensitive reversible hydrogels are herein defined as polymer-solvent systems that undergo a transition between a solution and a gel state in response to the external stimuli such as temperature, pH, ionic strength, solvent composition, sheer stress or a combination of these factors. A reversible stimuli-sensitive gel is one in which the transition is reversed upon reversal of the stimulus. A well known example of a reversible hydrogel is an aqueous solution of gelatin that is in a solution state at high temperatures (e.g. 80° C.) and forms a gel at lower temperatures (e.g., 20° C.). Other examples of reversible gels involve aqueous solutions of agarose and kappa-carrageenan that gel in response to the temperature change, and aqueous solutions of alginate that gel in response to the increased concentration of calcium ions. Reversible hydrogel systems are used in food and pharmaceutical industries as thickeners and suspending agents.

Some specific reversible gelling copolymers were also investigated as drug delivery systems and tissue engineering polymer matrices. High viscosity aqueous solutions containing 20% (or more) by weight of block copolymers of polyethylene oxide and polypropylene oxide, e.g. Poloxamer 407 and Pluronic F68 (Poloxamer 188) exhibit reverse thermal gelation. Solutions of Poloxamer 407 have been investigated for intraocular administration. Solutions containing 25% and 30% by weight Poloxamer 407 have been prepared and the force needed to inject them through a 25 gauge needle was investigated. It was concluded that a liquid-gel transition occurred inside the needle, due to the heat transfer between the needle walls and the surroundings. J. Juhasz et al., “Evaluation of the Injection Force of Poloxamer 407 Gels for Intraocular Administration,” 13 Pharm. Res., S-276 (1996).

In another example, a 25% by weight aqueous solution of Pluronic F68 was mixed with articular chondrocyte cells suspension at 4° C. and injected subcutaneously in nude and immunocompetent rabbit. In both cases, the cells entrapped in the copolymer formed tissue with histological appearance of hyaline cartilage. It was concluded that thermally reversible Pluronic F68 gel can serve as an effective injectable matrix for tissue engineering. C. A. Vacanti, et al., Proceedings of Tissue Engineering Society, Orlando, Fla. (1996).

An example of a pH-reversible hydrogel, investigated as an in situ gelling system for ophthalmic use is the aqueous solution of, a poly(acrylic acid)polymer, which undergoes a pH-mediated phase transition at concentrations above 0.1% by weight. The solution also contains hydroxypropyl methylcellulose, a viscosity enhancing agent. 13 Pharm. Res., Symposium Supplement (1996).

A new vehicle for topical and mucosal delivery, based on reversible gelation, was developed as an interpenetrating polymer network (IPN) of poly(acrylic acid) and a block copolymer of poly(ethylene oxide)/poly(propylene oxide). When heated from ambient to body temperature the network exhibited a significant viscosity increase from a viscous liquid to a gel-like consistency. It was concluded that at higher temperature, reduced release rates of active ingredients from the network were observed due to the increased viscosity of the IPN. E. S. Ron et al., “A New Vehicle for Topical and Mucosal Drug Delivery,” 13 Pharm. Res., S-299 (1996).

All gels containing the copolymers of poly(ethylene oxide)/poly(propylene oxide), i.e., Poloxamer 407, Pluronic F68 (Poloxamer 188), an IPN of poly(acrylic acid) and a block copolymer of poly(ethylene oxide)/poly(propylene oxide), and combinations thereof exhibit a limited, concentration dependent, stability of the gel state. The gels formed from these copolymers become liquids upon dilution (as for example due to the dilution with body fluids after peritoneal injection). Additionally, all the above examples of reversible hydrogels exhibit high initial viscosity in a liquid state, i.e., before the gelling transition.

The U.S. Pat. No. 5,262,055 to Bae et al. discusses an artificial pancreas utilizing reversible gels based on NiPAAM and its copolymers. These polymers and copolymers do not reverse upon dilution and they have a lower initial viscosity. However, the NiPAAM homopolymer described in Example 1 of Bae et al. forms a dense gel with minimal water content (i.e. exhibits substantial syneresis).

Polymers exhibiting phase transitions in water have many potential uses for drug delivery as stated in “Graft Copolymers that Exhibit Temperature-Induced Phase Transitions Over a Wide Range of pH,” G. Chen, A S Hoffman, 373 Nature, 49-52 (1995). In this paper, the authors further describe a temperature sensitive polymer that phase separates with a change in temperature or pH. Chen and Hoffman use graft copolymers having side chains of a temperature sensitive homopolymer, the oligo-N-isopropylacrylamide, grafted onto a pH sensitive backbone homopolymer of acrylic acid. The authors describe the phase separation of the graft copolymer investigated by a cloud point determination in dilute solutions. However, a dilute solution cannot produce a reversible gelation of these graft copolymers. Chen and Hoffman also mention random copolymers of N-isopropylacrylamide and acrylic acid as exhibiting a phase separation, however, there is no description of the intention to study the possibility of reversible gelation in more concentrated solutions of these random copolymers.

### SUMMARY

Disclosed herein are therapeutic agent carrier compositions that include a therapeutic agent carrier comprised of a stimulus sensitive gel or fibrin, or combinations thereof, and a therapeutic agent.

In some cases the stimulus sensitive gel is a thermally reversible gel or thermally reversible gelling copolymer that is a random copolymer of an [meth-]acrylamide derivative and a hydrophilic comonomer, wherein the random copolymer is in the form of a plurality of linear chains having a plurality of molecular weights greater than or equal to a minimum gelling molecular weight cutoff. The thermally reversible gelling copolymer is enhanced either by combining it with a therapeutic agent in an aqueous solution containing the thermally reversible gelling copolymer, and/or by grafting the thermally reversible gelling copolymer to a biodegradable backbone. The stimulus sensitive gel also can be selected from biodegradable polymers, for example polysaccharides, polypeptides, and combinations thereof; cellulose derivatives including but not limited to hydroxypropylmethyl cellulose; other polymers such as agar, gelatin, chitosan, alginate in combination with a slow gelling agent, for example, calcium sulfate, and combinations thereof.

For example, in some cases the stimulus sensitive gel is formed from a thermogelling biodegradable aqueous polymer solution, which includes a biodegradable polymer solution that includes a polyethylene glycol (PEG) block and a biodegradable polyester block linked to form a polymer.

In other cases the stimulus sensitive gel is formed from an enzymatically degradable polypeptide polymer. The peptide bonds in the polypeptide polymer are more stable against hydrolysis than are the ester bonds in PEG/PLGA polymer systems, thereby providing superior storage stability. Thus, the polypeptide carrier can be stored as a water solution, requiring no reconstitution. In some cases the polypeptide delivery system includes a biodegradable polymer having a biodegradable polypeptide block linked to a second polymer block to form a graft or linear polymer.

In some cases the carrier is a protein matrix known as fibrin formed by the reaction of fibrinogen and the enzyme thrombin. Fibrinogen is a naturally occurring protein which, when combined with enzyme thrombin, another naturally occurring protein, forms a bio-matrix known as fibrin.

In some instances the therapeutic agent is an anti-cancer agent, such as a radioactive or chemotherapy agent. In other cases the therapeutic agent is an anti-biotic agent. In some cases where the therapeutic agent is a radioactive agent, the radioactive agent includes an aqueous insoluble or confined radioisotope. In certain instances an aqueous insoluble radioisotope is a radioisotope insoluble salt such as, for example, 90-yttrium phosphate, 223-radium sulfate, and/or combinations thereof. In particular cases the radioactive agent including the radioisotope insoluble salt is hydrothermally synthesized, for example, in some embodiments particles of yttrium phosphate including 90-yttrium phosphate as an active agent are hydrothermally synthesized. In certain embodiments, such a radioactive agent is synthesized as, or suspended in solution phase to form, a colloid. For example, in particular embodiments, hydrothermally synthesized particles of yttrium phosphate including 90-yttrium phosphate are suspended in a continuous phase to form a colloid. In other particular embodiments, hydrothermally synthesized particles of yttrium phosphate including 90-yttrium phosphate are synthesized in a colloid and need not be subsequently suspended to form a colloid.

Methods of making therapeutic agent carriers also are disclosed. For example, therapeutic agent carriers may be made by hydrothermally synthesizing a radioactive agent including a radioisotope insoluble salt, suspending the salt in a colloid, and mixing the colloid with a therapeutic agent carrier such as a stimulus-sensitive gelling polymer, fibrin, or combinations thereof.

Further disclosed are methods of treating diseases such as cancer with therapeutic agent carrier compositions. For example, cancer is treated by applying a therapeutic agent carrier composition including a yttrium-90 colloid to the margins of a resected tumor site in an amount effective to kill vestigial cancer cells.

### BRIEF DESCRIPTION OF THE DRAWINGS

FIG. 1 is a photomicrograph of hydrothermally synthesized YPO₄ particles where the Y³⁺:EDTA:PO₄³⁻ reactant concentration ratio was 1:1:4 at a Y³⁺ concentration of 0.008M.

FIG. 2 is a graph illustrating the EDTA and PO₄³⁻ concentrations at which YPO₄ particle formation (precipitation) occurs and no particle formation occurs for a constant yttrium concentration of 0.008 M.

FIG. 3 is a graph illustrating the influence of reactant concentration on YPO₄ particle size for the Y³⁺:EDTA:PO₄³⁻ reactant ratios 1:1:4 and 1:1:3.

FIG. 4 is a graph illustrating the average mean diameter and standard deviation (shown by the I marks within the data columns) of particles for seven samples of hydrothermally synthesized ⁹⁰YPO₄ colloid as measured by a laser scanning particle size analyzer (LSPSA).

FIG. 5 is a depiction of a random copolymer of poly(N-isopropylacrylamide-co-acrylic acid) (NiPAAm/AAc), where n and m denote sequences of NiPAAm and AAc (respectively) that are of random length and are randomly distributed along the copolymer chain.

FIG. 6 is a bar graph of water retention in the gel versus initial copolymer concentration in the gelling solution.

FIG. 7A depicts a lymph node sectioned after the injection of thermally reversible copolymer/dye solution.

FIG. 7B depicts another lymph node sectioned after the injection of the dye solution alone.

FIG. 8 is a schematic representation of the synthesis of PEG-g-PLGA.

FIG. 9 is NMR spectra of intermediate and final products of PEG-g-PLGA synthesis.

FIG. 10 is a GPC chromatogram of polymers showing progress of reactions.

FIG. 11 is a Cryo-Transmission Electron Microscope (TEM) image showing micelle formation of the PEG-g-PLGA polymer at a concentration of 1 wt % in water at 23.7° C.

FIG. 12A is a graphical representation of UV spectra showing the formation of core-shell structure of polymers in water at 20° C. where diphenyl-1, 3, 5-hexatriene (DPH) concentration was fixed at 4 μM and polymer concentration varied according to key legend.

FIG. 12B is a graphical representation of the CMC determination by extrapolation of the difference in absorbance at 377 and 392 nm.

FIG. 13 is a graphical representation of real part (n′) of complex viscosity and elastic modulus (G′) of 22 wt % PEG-g-PLGA aqueous solutions as a function of temperature.

FIG. 14 is a phase diagram of PEG-g-PLGA aqueous solution.

FIG. 15 is a graphical representation of the calculation of enthalpy of solution-to-gel (sol-to-gel) transition of PEG-g-PLGA aqueous solutions.

FIG. 16 is a graphical representation of the ¹³C-NMR spectra of 22% by weight PEG-g-PLGA in D₂O as a function of temperature.

FIG. 17 is a graphical representation of the storage modulus of PLGA-g-PEG as a function of temperature and concentration.

FIG. 18A is a graphical representation of a rheological study of 25% by weight PLGA-g-PEG copolymer aqueous solutions wherein the real part (η′) of complex viscosity of the copolymer solution was measured as a function of temperature.

FIG. 18B is a graphical representation of a rheological study of 25% by weight PLGA-g-PEG copolymer aqueous solutions wherein the storage modulus (G′) of the copolymer solution was measured as a function of temperature.

FIG. 19 is a ¹³C-NMR (75 MHz) spectra of 25% by weight PLGA-g-PEG copolymer in D₂O as a function of temperature wherein the zoom spectra (˜73 ppm) are shown at left.

FIG. 20 is a deuterium NMR spectra showing reversibility of the sol-gel transition.

### DETAILED DESCRIPTION

As used herein, the term “stimulus sensitive gel” is a polymer solution that gels upon a change in stimulus. A stimulus includes but is not limited to temperature, pH, ionic strength, solvent composition, sheer stress or a combination of these factors. In some cases the gel is a reversible gel, more specifically, the gel is a random copolymer of a [meth-]acrylamide derivative with a hydrophilic comonomer. In other cases the stimulus sensitive gel includes a PEG block and a biodegradable polyester block linked to form a polymer. In still other cases the gel is an enzymatically degradable polypeptide polymer.

As used herein, the term [meth]-acrylamide refers to methacrylamide, acrylamide, or combinations thereof.

As used herein, the term therapeutic agent refers to any material or substance that is effective to treat any disease, illness, or other condition in a subject, such as a mammal, for example a human in need of such treatment. Effective to treat means that the therapeutic agent prevents, inhibits, or reduces the condition or its progress and/or prevents, inhibits, or reduces symptoms of the condition. In some cases the therapeutic agent is a radioactive agent, an anti-bacterial agent, or a chemotherapeutic agent.

As used herein, radioisotope refers to a radioactive isotope of a chemical element. The terms “radioisotope” and “radionuclide” are synonymous as used herein.

In some cases the therapeutic agent carrier is a radioactive agent carrier having a stimulus-sensitive gelling polymer or matrix-forming protein carrier, such as fibrin, mixed with a radioactive therapeutic agent including a radioisotope. When referring to radioactive therapeutic agents herein, the radioactive isotope may be designated in the name without referring to any non-radioactive isotopes or other materials that may be included in the radioactive therapeutic agent. For example a radioactive therapeutic agent may contain particles that include non-radioactive isotopes such as 89-yttrium phosphate in addition to radioactive isotopes such as 90-yttrium phosphate, but the radioactive therapeutic agent may be referred to herein simply as 90-yttrium phosphate for ease of reference.

In some examples the radioactive therapeutic agent includes a radioisotope that is an alpha and/or beta emitter with a short half-life. More specifically, in certain cases the radioisotope is selected from the group of yttrium-90, indium-111, radium-223, actinium-225, bismuth-212, bismuth-213, scandium-47, astatine-211, rhenium-186, rhenium-188, iodine-131, iodine-124, lutetium-177, holinium-166, samarium-153, copper-64, copper-67, phosphorus-32 and combinations thereof. Both low linear energy transfer (LET) and high linear energy (HLET) transfer radionuclides can be used. Alpha and beta particles have short path length in tissue, which indicates minimal irradiation of surrounding normal tissue.

In some cases the radioactive therapeutic agent is a confined radioisotope. The purpose of the radioactive confine is to minimize or prevent migration of the radioisotope to healthy tissue areas. The confined radioisotope may be confined, for example, by chelators or complexing agents, capsules, and combinations thereof. Useful isotope/chelator combinations are, for example, yttrium-90 or indium-111 with 1,4,7,10-tetraazacyclododecane-N,N′,N″,N′″-tetraacetic acid (DOTA), derivatives of DOTA; radium-223 with tetra-t-butyl-calix[4]arene-crown-6-dicarboxylic acid (TBBCDA), derivatives of TBBCDA; actinium-225 with 5,11,17,23-tetra-t-butyl-25,26,27,28-tetrakis(carboxymethoxy)-calix[4]arene (TBTC), derivatives of TBTC; and bismuth-212, bismuth-213 with 5,11,17,23,29,35-hexa-t-butyl-37,38,39,40,41,42-hexakis(carboxymethoxy)-calix[6]arene (HBHC), derivatives of HBHC, diethylenetriamine-pentaacetic acid (DTPA), or ethylenediaminetetraacetic acid (EDTA), derivatives of DTPA, and combinations thereof. The confined radioisotope can be glass beads and/or polymer beads.

In certain cases the radioactive therapeutic agent includes an insoluble salt that includes a radioisotope, such as 90-yttrium phosphate, 223-radium sulfate, and/or combinations thereof. In some embodiments the radioactive agent includes one or more particles formed of the insoluble salt. In particular cases the particles of the insoluble salt are hydrothermally synthesized, for example, particles including 90-yttrium phosphate are hydrothermally synthesized. In certain embodiments such particles are formed in solution as the disperse phase of a colloid by hydrothermal synthesis As used herein a colloid is a chemical system composed of a continuous medium (continuous phase) throughout which are distributed small particles, for example about 0.0001 μm to about 3 μm in size (the disperse phase).

Hydrothermal synthesis refers to the synthesis of products by reacting reagents in solution at temperatures and/or pressures above ambient temperature and/or pressure, such as by performing the reaction in a sealed vessel (generally known as a hydrothermal bomb) that may also be heated. The hydrothermal bomb may include a liner in which reagents are reacted so that the bomb can more easily be reused.

Hydrothermal synthesis of insoluble salt particles allows for control of particle shape and/or size. Controlling particle size and/or shape is useful for optimizing the retention of particles in a disclosed therapeutic agent carrier. Particles in a therapeutic agent carrier having diameters between about 0.2-1 μm generally show a superior retention in the carrier (when in vivo). In certain cases substantial amounts of the particles, such as 40% or more, or even 60-90% or more of the particles are substantially spherical in shape. When in a colloid such particles are referred to as monodispersed, that is they are particles of substantially uniform size (diameter) in a disperse phase. Uniform size and shape are not required, but these characteristics can aid in determining the amount of radioactive agent to administer to achieve a particular dosage of radiation to tissue in vivo. That is, the more uniform the particles are in size and/or shape the more consistent the radiation doses for a particular amount of particles because similar sized and/or shaped particles provide similar amounts of radiation.

In certain embodiments the radioactive therapeutic agent includes radioisotope insoluble salt particles that have an average diameter of from about 0.3 to about 3 μm or that have a mean diameter in the same range. In particular embodiments particles with an average and/or mean diameter of about 0.3 to about 0.8 μm are used. The particles are retained well in the disclosed therapeutic agent carriers comprising, for example, a gel or a protein-matrix, or combinations thereof.

Certain embodiments of hydrothermal syntheses of insoluble salt radioactive therapeutic agents, such as YPO₄ particles, include a complexing agent, such as a complexing agent comprising ethylene diamine tetraacetic acid (EDTA), to bind metal cations, such as Y³⁺, in solution, allowing the cations to exceed the saturation concentration without significant precipitation of the salt. During hydrothermal synthesis the EDTA releases the cations to react with anions, such as YPO₄³⁻, to form particles. In certain embodiments the particles formed are colloidal, that is, the particles form a disperse phase of a colloid in the continuous phase of the solution.

In certain embodiments, colloids including YPO₄ particles as the disperse phase are synthesized using EDTA, a yttrium (Y) source, and a phosphate (PO₄) source, all reacted in a hydrothermal bomb. The Y source may include yttrium chloride (YCl₃). In certain embodiments the yttrium chloride is ⁹⁰YCl₃. As another example, the yttrium source may be a combination of ⁹⁰YCl₃ and a non-radioactive Y source, such as ⁸⁹YCl₃, depending on the amount of radioisotope desired in the colloid and the amount of time expected to elapse from synthesis of the colloid until the colloid will be used therapeutically. One of ordinary skill in the art is readily able to determine the appropriate amount of radioactive Y source, for example ⁹⁰YCl₃ to use based on the desired radioactivity of the product.

The phosphate source in some embodiments includes a monosodium phosphate (NaH₂PO₄), disodium phosphate (Na₂HPO₄), phosphoric acid (H₃PO₄), and/or mixtures thereof. Other suitable phosphates also may be used. The concentration of the phosphate source may be, for example, from about 0.01 M to about 0.2 M. For example, Na₂HPO₄ at a concentration of from about 0.1 M to about 0.2 M may be used.

The reactants may be reacted in a Y:EDTA:PO₄ ratio of, for example, about 1:1:(3-6). The reaction may be performed in an acidic environment, that is, below about pH 7, for example, at a pH of about 1 to about 7. In certain embodiments the reaction is performed in a highly acidic environment, such as a pH of about 1 to about 3, while in other cases the environment is only slightly acidic, such as a pH of about 5 or 6 to about 6.5. In still other embodiments the reaction is performed in an environment of medium acidity, for example a pH of from greater than about 3 to about 5.

In some embodiments the hydrothermal bomb is heated to a temperature of from about 120° C. to about 180° C. for a period of about 3 hours to about 20 hours or more after or during mixture of the reactants. The bomb may be heated to, for example, about 150° C. for about 20 hours. After heating, the colloid product is cooled to room temperature. In some cases the colloid is air cooled for about 2 to about 6 hours. In other cases the colloid is allowed to cool over a longer period, such as from about 6 to about 20 hours or more. During cooling the colloid can be stirred (or otherwise mixed or agitated) to cool the colloid more quickly. For example, when cooling for only about 2 to about 6 hours the colloid may be stirred. In particular cases the colloid is sonicated in the hydrothermal bomb liner (or is otherwise treated to recover additional product from the liner). Filtering the colloid product yields a YPO₄ cake.

A variety of different YPO₄ particle sizes (as determined by measuring the average diameter or the mean diameter of the particles) can be produced by the disclosed hydrothermal synthesis methods. For example, particles having an average diameter, or a mean diameter in the range from about 0.2 μm to about 3 μm can be produced, as measured, for example, by a scanning electron microscope (SEM) or by a light scattering particle size analyzer (LSPSA). The size of the particles may depend on the concentrations of the reactants used. For example, in one embodiment, a reactant group comprising Y:EDTA:PO₄ at a ratio of 1:1:4 with a Y concentration of about 0.010 M, an EDTA concentration of about 0.01 M, and an NaH₂PO₄ concentration of about 0.01 M resulted in substantially all particles being substantially spherical in shape and having an average diameter of about 1-2 μm as measured by SEM (Japan Electron Optics Laboratory (JEOL) JSM-5900 LV). The size and shape of the particles were determined by visually examining SEM photos and comparing the particles with a scale bar. In another embodiment, the same Y:EDTA:PO₄ ratio was utilized but the Y concentration was about 0.008 M. This reactant mixture resulted in substantially spherical particles having an average diameter of about 1 μm, as measured by SEM. In another embodiment, the same Y:EDTA:PO₄ ratio of 1:1:4 was used and the Y concentration was about 0.004 M. Such mixture resulted in agglomerates having an average diameter of about 0.3 μm, as measured by SEM. An agglomerate is a clump of small particles (as opposed to monodispersed spheres). Table 1 describes particular embodiments of hydrothermal reactant conditions and resulting YPO₄ particles.

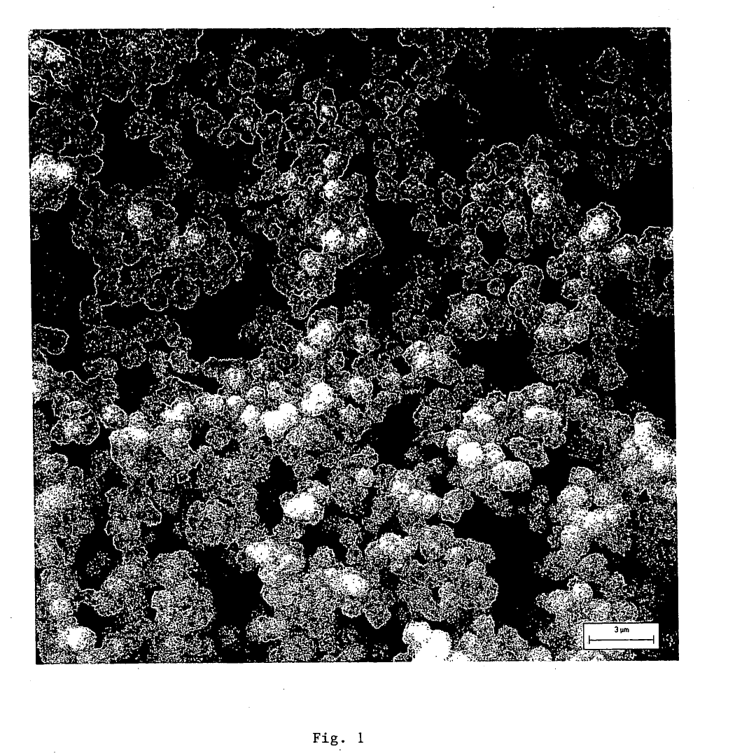

TABLE 1
Hydrothermal reactant conditions and
descriptions of the average diameters
and physical characteristics of resulting
YPO₄ particles.

| | | pH of | | |
|---|---|---|---|---|
| | | Reactant | | |
| | | Mixture | | Description of |
| Y³⁺:EDTA:PO³⁻₄ | PO₄³⁻ | (w/ | | particles (average |
| RATIO | Source | TEAOH) | Y³⁺ conc. | diameter/shape) |
| 1:1:4 | NaH₂PO₄ | 3 | 0.010 M | 1-2 μm |
| | | | | spheres/rough |
| | | | | surface, a small |
| | | | | amount have |
| | | | | necks |
| 1:1:4 | NaH₂PO₄ | 3 | 0.008 M | ˜1 μm |
| | | | | spheres/rough |
| | | | | surface, as |
| | | | | shown in FIG. 1 |
| 1:1:4 | NaH₂PO₄ | 3 | 0.004 M | <0.3 μm |
| | | | | agglomerates |
| 1:1:3 | NaH₂PO₄ | 3 | 0.010 M | 1-3 μm |
| | | | | spheres/very |
| | | | | round, smooth |
| | | | | surface |
| 1:1:3 | NaH₂PO₄ | 3 | 0.008 M | <0.5 μm |
| | | | | agglomerates |
| 1:1:3 | NaH₂PO₄ | 3 | 0.004 M | Mixture of |
| | | | | morphologies, |
| | | | | e.g., 3 μm spheres |
| | | | | & <0.5 μm |
| | | | | agglomerates |

Examples 1-4 show particular embodiments of the hydrothermal synthesis method embodiments that form particular embodiments of the disclosed YPO₄ particles and colloids. The examples are not limiting and are illustrative only.

### EXAMPLE 1

Although the reactants can be mixed together in any order, in this specific example the reactants were mixed in the order discussed. An embodiment of the disclosed YPO₄ particles was synthesized in a colloid by combining about 8 ml of about 0.1 M EDTA and about 52 ml deionized water in a TEFLON liner of a hydrothermal bomb. The reactants were mixed thoroughly. About 8 ml of about 0.1 M YCl₃ was added to the water and EDTA mixture and thoroughly mixed. About 32 ml of about 0.1 M NaH₂PO4 was added to the water, EDTA, and YCl₃ mixture and thoroughly mixed. The pH of the resulting mixture was adjusted to about 3 using about 0.1 M tetraethyl ammonium hydroxide (TEAOH). The liner was placed in a hydrothermal bomb. The hydrothermal bomb was heated in an oven to about 150° C. for about 3 hours. The mixture was cooled in the bomb to room temperature. The resulting product was a colloid including monodispersed YPO₄ particles as the disperse phase. An SEM view of the YPO₄ particles (not as suspended in the colloid because the colloid product was dried to allow microscopy) is shown in FIG. 1. As can be seen, a substantial amount of the resulting YPO₄ particles were substantially spherical. The particles had an average diameter of about 1 μm, as measured by SEM. The yield of YPO₄ colloid particles from this process was about 15% as determined by measuring the mass of solids used in the reaction and the mass of the final YPO₄ product.

### EXAMPLE 2

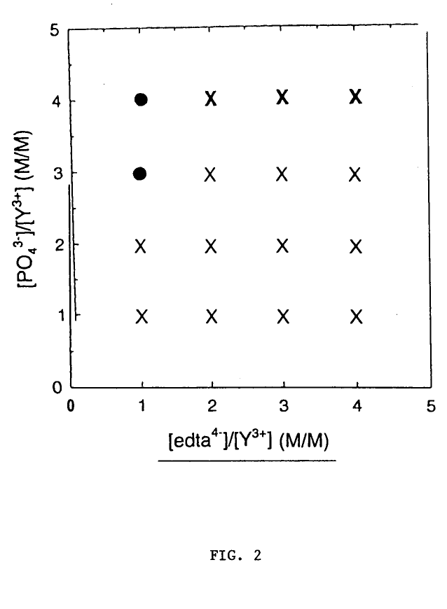

In this example the synthesis described in Example 1 was performed for 16 different combinations of YCl₃, NaH₂PO₄, and EDTA using a constant Y³⁺ concentration of 0.008 M and NaH₂PO₄ and EDTA each in concentrations of 0.008 M, 0.016 M, 0.024 M, and 0.032 M such that each possible combination of NaH₂PO₄ and EDTA concentrations was used with the 0.008 M Y to synthesize YPO₄. The reactant concentrations at which YPO₄ particles precipitated (that is, were formed) are shown in FIG. 2. As can be seen, particle formation occurred under most conditions, although no particles formed in this example where the EDTA concentration was 0.008 and the NaH₂PO₄ concentration was 3 or more times higher than the EDTA concentration.

### EXAMPLE 3

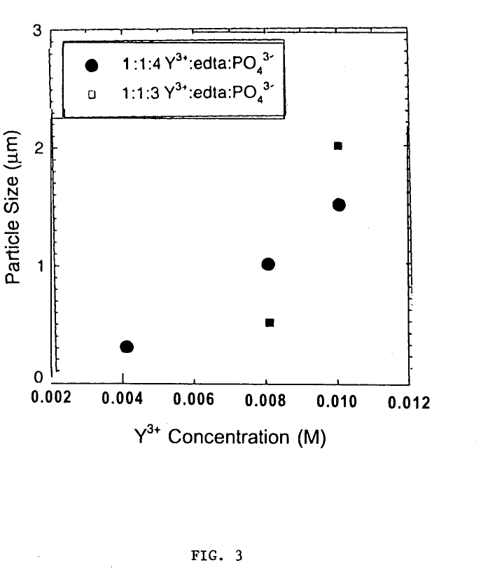

In this example the synthesis described in Example 1 was performed at Y³⁺ (from YCl₃) concentrations of 0.004 M, 0.008 M, and 0.010 M and Y³⁺:EDTA:PO₄³⁻ ratios of 1:1:4 and 1:1:3. As can be seen in FIG. 3, the higher the Y³⁺ concentration the greater the average diameters of the resulting particles.

### EXAMPLE 4

In this example a different method (from the methods described in Examples 1-3) of hydrothermal synthesis of YPO₄ was used that included using a Y source including ⁹⁰YCl₃ to yield radioactive particles. This embodiment of the disclosed method significantly increased yields of YPO₄ particles and produced monodispersed microspheres having a smaller mean diameter than the average diameters of the particles formed in Examples 1-3. In this example, the reaction was performed with a 50% higher concentration of Na₂HPO₄ than in Example 1. Also, the amount of PO₄ used was 50% higher than in Example 1 (that is, a Y:EDTA:PO₄ ratio of about 1:1:6). The reaction was performed at a higher pH and heated for a substantially longer period of time than in Example 1. This example method included a shorter cooling period during which the mixture was stirred. Sonification of the resulting colloid in the hydrothermal bomb liner was also performed. This resulted in a yield of about 70%. In cases where radioactive YPO₄ is produced (such as ⁹⁰YPO₄), the yield is determined by measuring the difference of the radioactivity of the reagents to the radioactivity of the final ⁹⁰YPO₄ product.

As with the previous examples, the reactants can be mixed in any order, although in this particular example they were mixed in the order described below. A colloid including ⁹⁰YPO₄ particles was synthesized by combining about 8 ml of 0.1 M EDTA and about 52 ml ultrapure (UP) water in a TEFLON hydrothermal bomb liner and mixing thoroughly with a magnetic stir bar. About 8 ml of about 0.1 M ⁸⁹YCl₃ was added to the EDTA and water. About 32 ml of about 0.15 M Na₂HPO₄ was added to the EDTA, water, and ⁸⁹YCl₃. About 900 μL of ⁹⁰YCl₃ stock solution (about 20 millicuries of ⁹⁰YCl₃ dissolved in 0.05 M hydrochloric acid (HCl)) was added to the mixture. The mixture was mixed continuously during these steps. The pH of this mixture was adjusted to about 6.5 by adding about 0.05 M HCl.

The mixture, in the liner, was placed into the hydrothermal bomb in an oven at about 150° C. for about 20 hours. After removal of the hydrothermal bomb from the oven, the mixture was stirred with a stir bar on a magnetic stirring plate (e.g., at a speed of about 5-9) for about 4 hours while being air-cooled.

The TEFLON liner was removed from the bomb and placed in a 1 L glass beaker filled with water. The liner and beaker were placed in an ultrasonic bath for about 30 minutes. The YPO₄ colloid was filtered using a 0.2 μm nylon membrane filter. The TEFLON liner was rinsed with 100 ml UP water and sonicated in an ultrasonic bath for about 10 minutes. This rinse water was filtered. The resulting YPO₄ particle cake was rinsed with 100 ml UP water 3 times. The cake was placed in a 20 ml scintillation vial with a small stirring bar. About 1 ml of phosphate buffered saline (PBS) solution was added to the vial and the PBS and YPO₄ particles were stirred for 15 minutes to suspend the particles saline solution and form a colloid. The yield of YPO₄ colloid from this process was about 70%.

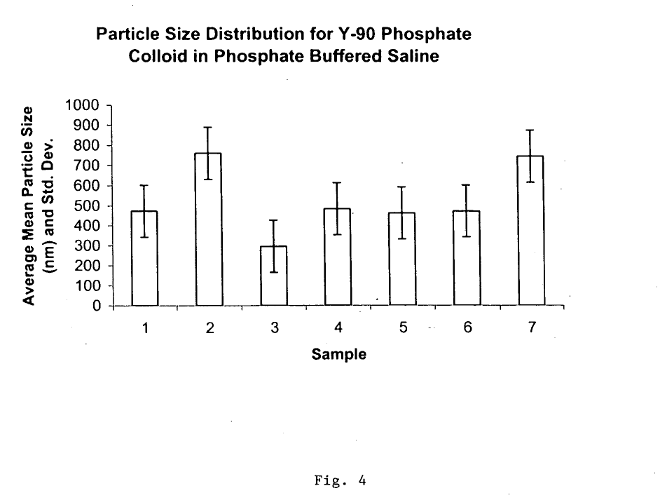

This embodiment resulted in particles having smaller sizes than the particles formed in the embodiments described in Examples 1-3. The mean particle size for colloidal particles made by this process was determined by making seven different colloidal suspensions. A portion of each colloidal suspension (about 3-4 mg) was further suspended in about 4-5 g of a PBS solution. Each suspension was analyzed four to six times with a Laser Scattering Particle Size Analyzer (LSPSA) by Beckman Coulter to determine the mean sphere diameter. The LSPSA software calculated the mean diameter and reported the size distribution of the suspended particles. As illustrated in FIG. 4, the average mean sphere diameter for colloids made by this process disclosed in this example ranges from about 0.3 to about 0.8 μm.

The amount of ⁹⁰YPO₄ colloid particles or other radionuclides used in the therapeutic agent can be determined by those of ordinary skill in the art of radiation dosimetry given the disclosure herein that retention of the radionuclide in the therapeutic agent carrier and the retention of the carrier in and/or near the tumor is very high. Some of the factors one of skill in the art would take into consideration include, for example, the half life of the radionuclide, the number of transformations, the type of particles emitted, the distance traveled by the particles, the estimated fraction of radiation adsorbed locally, and the mass of the target tumor.

In typical external beam rotation radiation therapy a radiation absorbed dose (rad) of about 6000 to about 8000 rad (centigray) is used to treat a target (such as a tumor) because higher doses would exceed tolerance of limiting normal tissue.

However, due the to location of the radionuclide achieved by the methods described herein and the superior retention of the radionuclide in the disclosed therapeutic carrier agents, much higher radiation absorbed doses can be used, for example from about 5000 to about 1,000,000 rad or more. The upper limit of the radiation dose delivered with the compositions and methods described herein depends on the concentration of the radioisotope achieved. An objective in radiation therapy is to achieve a high therapeutic index, which is the ratio of the absorbed dose to the target tissue relative to the absorbed dose to the critical limiting normal organ or tissue in the patient that is not specifically targeted. With the compositions and methods described herein, a maximum theoretical therapeutic index may be achieved. For example, the radiation dose to a target tissue may be doubled merely by doubling the concentration of the radionuclide in the therapeutic agent carrier prior to injection. The dose to target tissue may be increased to the point at which additional radiation dose results in no additional therapeutic benefit—the maximum theoretical therapeutic index.

In other examples the therapeutic agent carrier is a chemotherapy agent carrier. A chemotherapeutic agent includes, for example, an alkylating agent or nitrogen mustard (mechlorethamine, nitrosoureas, melphalan, chlorambucil, cyclophospharnide, infosfamide), and antimetabolite (methotrexate, 5-fluorouracil, cytosine arabinoside, 6-mercaptopurine, 6-thioguanine, fludarabine, pentosatin), and antitumor antibiotic (actinomycin D, anthracyclines such as doxorubicin, bleomycin, mitomycin C, mithramycin), a plant alkaloid (vincristine, vinblastine, etoposide or VP-16), a hormone or a hormone antagonist (synthetic estrogen diethylstilbestrol DES, gonadotropin-releasing hormone, corticosteriods, tamoxifen or NOLVADEX, raloxifen or EVISTA), or any other chemotherapy agent such as cisplatin cis-diamminedichloro-platinum II, docetaxel or TAXOTERE, and capecitabine or XELODA. The chemotherapy agent can be prepared as a soluble matrix to enhance slow-release from the stimulus sensitive gel, such as fibrin, into the target tissue.

A therapeutic agent carrier including a stimulus-sensitive gelling polymer is one that forms a gel without substantial syneresis when the stimulus-sensitive polymer is in an aqueous solution. Syneresis is defined as a substantial amount of water expelled from a polymer matrix upon gelation. Substantial syneresis is more than about 10% water (by weight) expelled from the polymer matrix. Syneresis of less than about 10%, or less than about 5% or less than about 2% is quite useful. Substantially no syneresis is syneresis of less than about 2%.

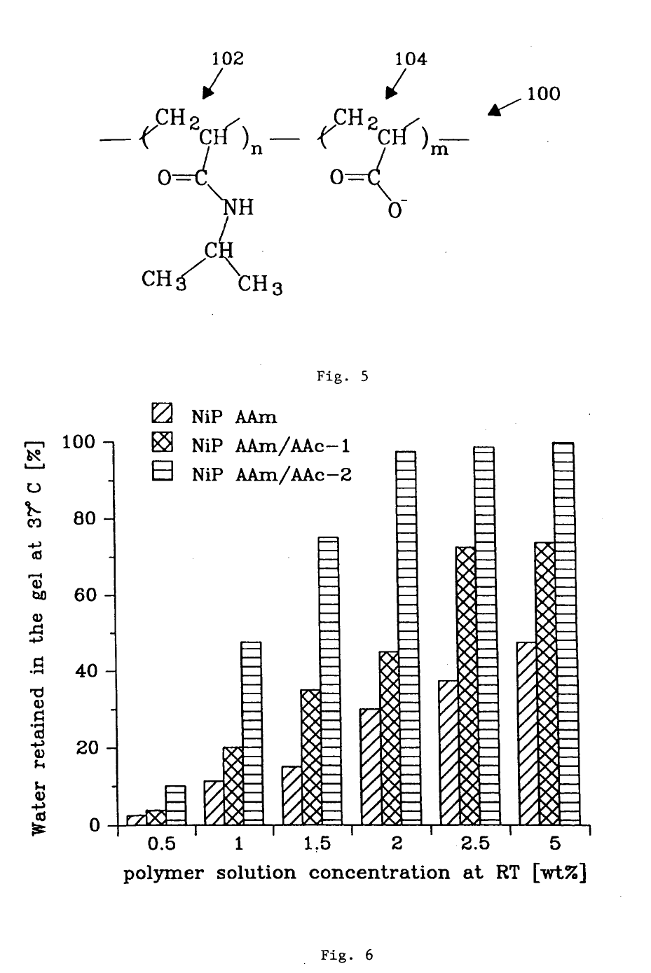

In some examples the stimulus sensitive gel is a thermally reversible copolymer that is a linear random copolymer of an [meth-]acrylamide derivative and a hydrophilic comonomer wherein the linear random copolymer is in the form of a plurality of linear chains having a plurality of molecular weights greater than or equal to a minimum gelling molecular weight cutoff. The minimum gelling molecular weight cutoff is at least several thousand and is preferably about 12,000. The presence of a substantial amount of copolymer or polymer chains having molecular weights less than the minimum gelling molecular weight cutoff results in a milky solution that does not gel. Further, the amount of hydrophilic comonomer in the linear random copolymer is preferably less than about 10 mole %, more preferably less than about 6 mole % and most preferably about 2-5 mole %. The structure of linear chains is not cross-linked. Moreover, the linear random copolymer structure is one in which a linear chain 100 is shared by randomly alternating portions of the [meth-]acrylamide derivative 102 and the hydrophilic comonomer 104 as depicted in FIG. 5.

The [meth-]acrylamide derivative is an N,N′-alkyl substituted [meth-]acrylamide including but not limited to N-isopropyl[meth-]acrylamide, N,N′-diethyl[meth-]acrylamide, N-[meth-]acryloylpyrrolidine, N-ethyl[meth-]acrylamide, and combinations thereof.

The hydrophilic comonomer is any hydrophilic comonomer that co-polymerizes with the [meth-]acrylamide derivative. Preferred hydrophilic comonomers are hydrophilic [meth-]acryl-compounds including but not limited to carboxylic acids, [meth-]acrylamide, hydrophilic[meth-]acrylamide derivatives, hydrophilic [meth-]acrylic acid esters. The carboxylic acid may be, for example, acrylic acid, methacrylic acid and combinations thereof. The hydrophilic acrylamide derivatives include but are not limited to N,N-diethyl[meth-]acrylamide, 2-[N,N-dimethylamino]ethyl[meth-]acrylamide, 2-[N,N-diethylamino]ethyl[meth-]acrylamide, or combinations thereof. The hydrophilic [meth-]acrylic esters include but are not limited to 2-[N,N-diethylamino]ethyl[meth-]acrylate, 2-[N,N-dimethylamino]ethyl[meth-]acrylate, and combinations thereof.

The stimulus-sensitive polymer can be mixed with an aqueous solvent to form a stimulus-sensitive gelling solution, or reversible gelling solution. The aqueous solvent includes but is not limited to water and aqueous salt solutions. In certain cases the salt solution is a phosphate buffered saline solution for medical use.

In certain examples the thermally reversible polymer is made by performing the steps of:

(a) mixing an [meth-]acrylamide derivative with a hydrophilic comonomer in a reaction solvent with an initiator forming a reaction mixture;

(b) polymerizing the reaction mixture and forming a first linear random copolymer having a plurality of linear chains having a plurality of molecular weights; and

(c) isolating and purifying the polymerized first linear random copolymer and obtaining a second linear random copolymer having a plurality of molecular weights greater than or equal to a minimum gelling molecular weight cutoff.

The alternatives for the [meth-]acrylamide derivative and the hydrophilic comonomer have been set forth above and are not repeated here.

The reaction solvent can be aqueous or non-aqueous. The preferred aqueous solvent is simply water. In some cases, the aqueous solvent is a salt solution. A non-aqueous solvent can be a hydrocarbon including but not limited to oxygenated hydrocarbon solvent, for example dioxane, chlorinated hydrocarbon solvent, for example chloroform, an aromatic hydrocarbon, for example benzene. Precipitation of the polymer occurs during polymerization in benzene. Dioxane is a preferred solvent because there is no precipitation during copolymerization thereby imparting greater uniformity of composition of the random copolymer (NiPAAM/AAc).

The amount of aqueous solvent with respect to [meth-]acrylamide derivative is preferably about 80% (by weight), but can range from about 30% to about 98%. The amount of non-aqueous solvent with respect to the [meth-]acrylamide derivative is preferably about 80% but can range from about 30% to about 98%.

The initiator can be any free radical initiator compatible with the [meth-]acrylamide derivative. In some examples the initiator is 2,2′-azobis-isobutyrolnitrile (AIBN). The amount of the initiator with respect to the reaction mixture of solvent and polymer is preferably about 0.1% (by weight), but may range from about 0.01% to about 2%.

In some cases a reversible gelling solution is made by mixing the thermally reversible polymer with an aqueous solution. The amount of aqueous solution with respect to polymer is from about 70% (by weight) to about 99%, preferably about 98% for NiPAAm/AAc to achieve a nonresorbable reversible gel with substantially no syneresis. The aqueous solution is preferably a salt solution.

In addition to the nonresorbable reversible gel composed of a linear random copolymer of N-isopropyl[meth-]acrylamide and [meth-]acrylic acid, a biodegradable (resorbable) copolymer exhibiting similar gelation properties is obtained by grafting of the oligo [meth-]acrylamide derivative side chains on a biodegradable homopolymer backbone of, e.g., poly(amino acid). In certain cases oligo [meth-] acrylamide derivative side chains include N,N-alkyl substituted [meth-] acrylalmide derivatives, linear random copolymer of [meth-]acrylamide derivative and hydrophylic comonomer, and combinations thereof. Techniques of grafting of oligo-N-isopropyl[meth]acrylamide side chains on a nonbiodegradable pH-sensitive homopolymer backbone are described (Chen and Hoffman). The technique(s) of Chen and Hoffman were used to graft the oligo-N-isopropyl[meth-]acrylamide side chains on an alternative biodegradable homopolymer backbone such as poly(amino acid). The first step of the synthesis is either the free-radical homopolymerization or the random copolymerization of the oligo-N-isopropyl[meth-]acrylamide side chains by free radical homopolymerization using an amino-terminated chain transfer agent, for example 2-aminoethanethiol hydrochloride. The next step is the coupling of the amino-terminated macromer to the carboxyl moieties of the biodegradable backbone using the activation reagent, e.g., dicyclohexyl carbodiimide. Other biodegradable backbones such as poly(phosphazenes) and poly(caprolactone) also can be grafted with the oligo-N-isopropyl[meth-]acrylamide side chains using similar synthetic techniques. The reaction solvent is non-aqueous, preferably a hydrocarbon, for example chloroform, dichloromethane, N,N′-dimethylformamide or combinations thereof.

### EXAMPLE 5

An experiment was conducted to demonstrate synthesis and thermoreversible gel formation of poly(N-isopropylacrylamide-co-acrylic acid)(NiPAAm/AAc). The linear high molecular weight NiPAAm/AAc copolymers containing different amounts of AAc were synthesized by a free radical copolymerization.

The [meth-]acrylamide derivative was N-isopropylacrylamide (NiPAAm) (Fisher, Co.) that was recrystallized from hexane before use. The initiator 2,2′-azobis-isobutyronitrile (AIBN) (Eastman Kodak, Co.) was recrystallized from methanol. The hydrophilic comonomer was acrylic acid (AAc) (Aldrich Co.) that was purified before use by vacuum distillation at 39° C./10 mmHg. The reaction solvent, dioxane, HPLC grade (Aldrich Co.) was used as received. The mixture of [meth-]acrylamide derivative, initiator, hydrophilic comonomer, and solvent formed the reaction mixture.

The molar feed ratio of NiPAAm to AAc was varied as 99:1, 98:2 and 97:3. The copolymerization was carried out in dioxane (80% by weight), with the amount of AIBN initiator of 1.219×10⁻³ mols/L. The reaction proceeded at 60° C. for 18 hours. The resulting copolymer solution was diluted with fresh dioxane and added dropwise to a ten-fold excess of diethyl ether producing copolymer precipitation. The precipitated copolymer was isolated by filtration and drying. The isolated copolymer was redissolved in acetone and reprecipitated into ten-fold excess diethyl ether. The final, essential step of purification involved dialysis of aqueous copolymer solution through 12,000-14,000 molecular weight cut off (MWCO) dialysis membrane. Dialysis removed the residual unreacted monomer and all copolymer fractions with molecular weights smaller than the MWCO of the dialysis membrane, resulting in a purified copolymer product. The purified copolymer product was further freeze dried.

The removal of molecular weights below 12,000 from the synthesized copolymers was confirmed by gel permeation chromatography. The removal of unreacted monomers was confirmed by nuclear magnetic resonance.

The lower critical solution temperature (LCST) of the synthesized copolymers was evaluated by the cloud point determination method. In this method, 1% (by weight) solutions of synthesized copolymers in phosphate buffered saline (PBS) were heated from 20° to 50° C. in 2-degree increments every 10 minutes and the absorbance at 450 nm was measured. The cloud point, corresponding to the LCST was determined as the temperature at the inflection point in the absorbance versus temperature curve. NiPAAm homopolymer exhibited an LCST at 32° C. Copolymerization with hydrophilic comonomers shifted the LCST to the physiological temperature range of 36°-38° C. NiPAAm/AAc copolymer containing 2 mole % of AAc exhibited the LCST at 37° C.

Thermally reversible gel formation was studied at 37° C. The freeze-dried copolymer was dissolved in PBS at different copolymer concentrations (0.5, 1.0, 1.5, 2.0, 2.5, and 5.0% by weight) forming copolymer solutions. The PBS was specifically 0.15M NaCl and 0.01 M phosphates KH₂PO₄ and Na₂HPO₄. The copolymer solutions were thermally equilibrated at 37° C. for 24 hours. The syneresis (amount of water expelled from the gel) was measured gravimetrically. Syneresis of thermoreversible hydrogels of N-isopropylacrylamide (NiPAAm) and its copolymers with acrylic acid (AAc) was affected by copolymer composition (0, 1, 2 mole % of AAc) and polymer concentration as shown in FIG. 6. In FIG. 6 the amount of water retained in the gel is plotted as a function of the initial copolymer concentration in solution (before gelling). It was unexpectedly discovered that the solution containing at least about 2% of the NiPAAm/AAc copolymer having at least about 2.0 mole % of AAc was able to produce a reversible gel exhibiting substantially no syneresis.

### EXAMPLE 6

An experiment was conducted to confirm the necessity of the minimum gelling molecular weight cutoff. A gelling polymer solution was made as in Example 5, but the solution was not dialyzed so that no low molecular weight species were removed. The result was a solution, milky in appearance, that did not form a gel.

In other embodiments the stimulus sensitive therapeutic agent carrier is fibrin. For example, in certain embodiments a radioactive therapeutic agent, such as a colloid including radioactive insoluble salt particles, for example, ⁹⁰YPO₄ particles, is mixed with fibrin. Fibrin is an elastic, insoluble protein matrix derived from the interaction of fibrinogen with thrombin in the presence of calcium, forming a fibrous network that aids in the coagulation of blood. Fibrin has been developed as a topical “adhesive.” For example, cryoprecipitated antihemophilic factor may be mixed with thrombin to prepare a topical fibrin sealant to aid in tissue apposition and surgical hemostasis. In other cases, fibronectin (a glycoprotein consisting of two polypeptide chains connected by two disulphide bridges) can be utilized rather than fibrin. A fibrin bandage can be constructed by lyophilizing fibrin with thrombin and fibrinogen layered on a vicryl mesh backing.

Fibrin adhesives are known in the art: see Spotnitz, et. al, The American Surgeon 53:460-462, 1987; Alving, et al, Transfusion, J Am. Assoc. Blood Banks, 35(9): 783-790, 1995; Loose and Haslam, Brit. J Radiol 71: 1255-1259, 1998. Whole blood is collected from a patient. Anticoagulant can be used to maintain the blood in liquid form. Plasma is separated from the blood. Plasma is separated from a fibrinogen-rich concentrate. Fibrin is prepared by initiation with batroxobin and endogenous thrombin. Typically thrombin is provided in a solution with calcium chloride. 120 mL of patient blood may yield about 4 to 5 mL fibrin sealant. A radioactive or chemotherapeutic therapeutic agent is added to the liquid fibrin sealant and mixed well to form a composition. In some cases the composition is then applied intraoperatively as a thin surface coating by an applicator directly to the tissue needing surface treatment. The applicator may be a brush, a squeeze-tube, blunt syringe, or other convenient device for placing the therapeutic fibrin adhesive onto a location to be treated. The fibrin therapeutic adhesive preferably remains sterile and pyrogen-free during preparation and administration.

In some cases fibrinogen and thrombin are kept separate until actually applied, for example, in separate barrels of a multi-barreled syringe. In these cases the therapeutic agent can be provided in the fibrinogen and/or in the thrombin, but typically is provided in the fibrinogen because of its greater viscosity.

Fibrin can be made from a patient's own blood (autologous fibrin) or may be made from donor blood (homologous fibrin) from either humans or animal blood plasma. Autologous fibrin is preferred because it is non-immunological and will not induce a rejection response, and it carries no outside pathogens. Also, its degradation products pose no health hazard to the patient. Use of homologous fibrin carries the potential of infecting the patient with human-donor infections (e.g., hepatitis B, aids) or animal-donor infections (e.g., Mad Cow disease).

In one example of a use for the fibrin therapeutic agent carrier composition, after surgical resection of a solid tumor, the remaining intact tissue is treated with a fibrin adhesive mixed with a radionuclide and/or a chemotherapeutic agent. The mixture is applied to the tissue surface with an applicator. Any remaining cancer cells are thereby irradiated or poisoned in situ. The short physical half-life of the therapeutic radionuclides will decay before biodegradation of the fibrin adhesive, and chemotherapy agents will be absorbed into cancerous tissue by that time.

While the simplest form of the therapeutic agent carrier including fibrin includes simply a fibrin carrier and a therapeutic agent intermixed with the fibrin carrier, in practice the therapeutic composition can be more complex. In some examples the fibrin carrier includes complexing agents, gels, collagens, and other agents. For example, radionuclide and chemotherapy agents can be chemically complexed with a targeting agent that binds specifically with fibrin proteins and polymers. For example, yttrium-90 may be complexed with the chelating agent 1,4,7,10 tetraazacyclo-dodecaine-N,N′, N″, N′″-tetraacetic acid (DOTA) or formed as a colloid (such as a ⁹⁰YPO₄ colloid) and linked to a fibrin-seeking peptide. An example of a fibrin-targeting agent is fibrinopeptide A.

Stimuli-sensitive polymer gels can be added to fibrin to help contain the radionuclide or chemotherapeutic agent within the fibrin adhesive. In a particular example the polymer gel is a copolymer of N-isopropylacrylamide with acrylic acid (poly NiPaam-co-AAC) in phosphate-buffered saline containing about 2 mole % acrylic acid synthesized by a free radical copolymerization process. In other examples the polymer gel is a thermogelling biodegradable aqueous polymer solution which includes a biodegradable polymer solution that includes a polyethylene glycol (PEG) block and a biodegradable polyester block linked to form a polymer, or the polymer gel can be an enzymatically degradable polypeptide polymer (both are discussed in more detail below).

In cases where fibrin is combined with a stimulus sensitive gelling polymer to form a therapeutic agent carrier the fibrin typically is about 60% to about 80% of the combination (by weight) and the gelling polymer is about 20% to about 40% of the combination (by weight), for example the combination can be 70% fibrin and 30% gelling polymer. In some cases fibrinogen and/or thrombin are both combined separately with a stimulus sensitive gelling polymer, for example in separate barrels of a multi-barrel syringe so that when applied from the syringe the fibrinogen and thrombin form fibrin. In these cases the fibrinogen and thrombin are combined with the stimulus sensitive gelling polymer in the same percentages as discussed above for fibrin/gelling polymer combinations.

Collagen protein can be added to enhance the binding of the therapeutic agent to the fibrin, to add bulk or filler, and to improve tissue adhesion.

The fibrin can contain an antifibrinolytic agent, such as aprotinin or epsilon amino caproic acid to help control the rate of biodegradation after placement.

Additionally, the fibrin can contain any member of the general class of antibiotics to help reduce the potential for infection after surgical closure of the wound.

Imaging agents can be added to the fibrin adhesive to aid in post-surgical external imaging of the implanted adhesive. Such agents include, for example, technetium-99m sulfur colloid for gamma-camera imaging, protein microbubbles for ultrasound imaging, and paramagnetic materials such as iron oxide for magnetic resonance imaging.

Dyes and colorants can be added to increase visual identification of treated areas.

Additional gelling polymers also are useful in the disclosed therapeutic agent carrier. Such gelling polymers can be used alone as carriers in combination with a therapeutic agent or may be combined with other carriers, for example fibrin, as discussed above. In some embodiments the polymer is a thermogelling, biodegradable polymer as disclosed in co-pending U.S. patent application Ser. No. 09/833,460, which is incorporated by reference herein. For example, the gel can be formed from a thermogelling biodegradable aqueous polymer solution that includes a biodegradable polymer solution, comprising a polyethylene glycol (PEG) block, and a biodegradable polyester block linked to form a polymer of a general structure comprising the formula of Aₙ(B), where n is greater than 2 and A is selected from the group of a polyethylene glycol block and a biodegradable polyester block, B is selected from the group consisting of a polyethylene glycol block and a biodegradable polyester block, and A is different from B, and an aqueous solution. In particular instances the thermogelling biodegradable aqueous polymer solution has an n between 3 and 10.

In some specific cases, the biodegradable polyester block is selected from the group of poly(DL-lactic acid), poly(L-lactic acid), poly(glycolic acid), poly(ε-caprolactone), poly(γ-butyrolactone), poly(α-valerolactone), poly(β-hydroxybutyric acid), and their copolymers or terpolymers. In certain instances the copolymers and/or terpolymers are selected from the group of poly(DL-lactic acid-co-glycolic acid), poly(L-lactic acid-co-glycolic acid), poly(ε-caprolactone-co-DL-lactic acid), copoly(ε-caprolactone-co-DL-lactic acid-glycolic acid).

In certain cases the biodegradable polyester blocks have a maximum molecular weight of 100,000 in order to accommodate solubility. In some cases the blocks have a molecular weight ranging from about 1,000 and 30,000, for example, from about 1,000 and 10,000.

In some cases the polyethylene glycol (PEG) blocks have an average molecular weight of between about 300 and 20,000, for example from about 500 and 10,000. A PEG block with a higher molecular weight than 10,000 is hard to be filter through glomeruli filtration.

The PEG polymer can be combined with an aqueous solvent (as has been discussed above) to form a solution. The percentage by weight of polymer in the solution can be from about 2% to about 70%, for example, from about 20% to about 30%. In even more specific examples the aqueous solution is about 21%-25% polymer (e.g., PEG-g-PLA) by weight. In some cases such a polymer solution gels upon heating. In certain instances the solution to gel transition temperature is from about 20° C. to about 40° C. Examples where the solution-to-gel transition temperature is about 30° C. are particularly useful because at human body temperature (about 37° C.) the solution transitions to a gel. In some examples the polymer gel is reversible and can transition back to solution upon cooling to a temperature below the solution to gel transition temperature.

Although U.S. patent application Ser. No. 09/833,460 contains a detailed description of multiple syntheses for the disclosed thermogelling, biodegradable polymer, for convenience, syntheses of certain of such polymers are disclosed below.

### EXAMPLE 7

This is an example of a synthesis of poly(lactic acid-co-glycolic acid) (PEG-g-PLGA). Materials for this synthesis include DL-lactide (Polyscience) and glycolide (Polyscience) recrystallized from ethyl acetate; glutaric anhydride (Aldrich), glutaric acid (Aldrich), stannous octoate (Aldrich), epoxy terminated polyethylene glycol (m.w.:600; Polyscience), poly(ethylene glycol m.w.:1000; Aldrich), and 1,6-diphenyl-1,3,5-hexatriene (DPH; Aldrich). The following are examples of steps for the synthesis. A schematic representation of this synthesis is shown in FIG. 8.

PEGs (m.w.=1000, 38.28 g, 38.28 mmole) were dissolved in 90 ml toluene. Toluene was then distilled off to a final volume of 50 ml to remove water by azeotropic distillation. Carboxylic acid terminated PEG (CPEG) was prepared by reacting PEG with excess amount of glutaric anhydride in the presence of catalytic amounts of glutaric acid. Glutaric anhydride (7.255 g, 80.39 mmole) and glutaric acid (0.042 g, 0.40 mmole) were added and the reaction mixture was stirred at 120° C. for 6 hours. The chemical shifts (ppm) in the spectra for this product were 1.9 (central methylene of glutarate), 2.4 (methylene of glutarate next to carbonyl group), 3.6 (ethylene of PEG), and 4.2 (methylene of PEG connected to glutarate). There were one to one area ratio of the peaks at 1.9 ppm and 4.2 ppm indicating the quantitative end group functionalization. Diethyl ether was added to the reaction mixture to precipitate out the carboxylic acid terminated PEG (CPEG). The product was placed under high vacuum (˜10⁻³ mm Hg) for 48 hours to remove the residual solvent.

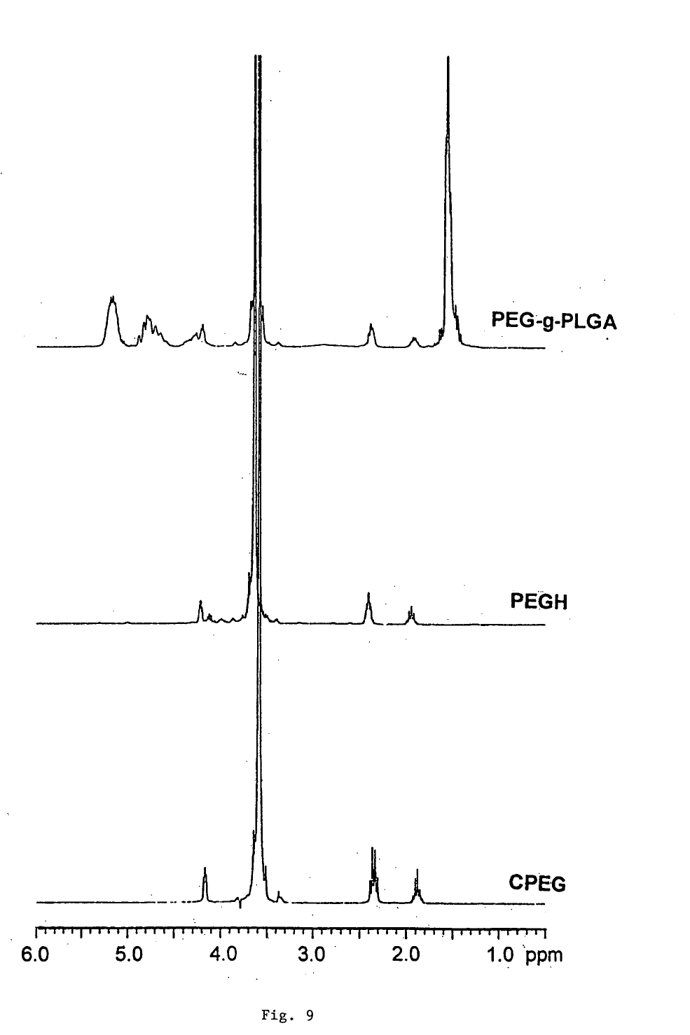

Epoxy terminated PEG (EPEG) (m.w.=600, 5.619 g, 9.36 mmole) was reacted with CPEG (11.50 g, 9.36 mmole) in toluene at 120° C. for 24 hours to prepare PEG with pendant hydroxyl groups (PEGH) along the PEG backbone. The weight average molecular weight (M_{w}) and polydispersity index (PDI) of resulting PEGH, which were determined by GPC, was 3000 and 1.3 relative to polystyrene standards. There were peaks at 1.9 ppm and 2.4 ppm come from glutarate (FIG. 9). There were peaks at 3.6 ppm and 4.3 ppm from PEG (FIG. 9). There were also small overlapped peaks at 3.4 to 4.2 ppm of PEGH from the connecting methylene or methine moieties between CPEG and EPEG (FIG. 9).

DL-lactide (19.2 g, 133.3 mmole) and glycolide (6.4 g, 55.1 mmole) were polymerized in situ on the preformed PEGH backbone at 130° C. for 24 hours, using stannous octoate (76 μL, 0.187 mmole) as a catalyst. The graft copolymers were precipitated into excess ethyl ether and the residual solvent was removed under vacuum.

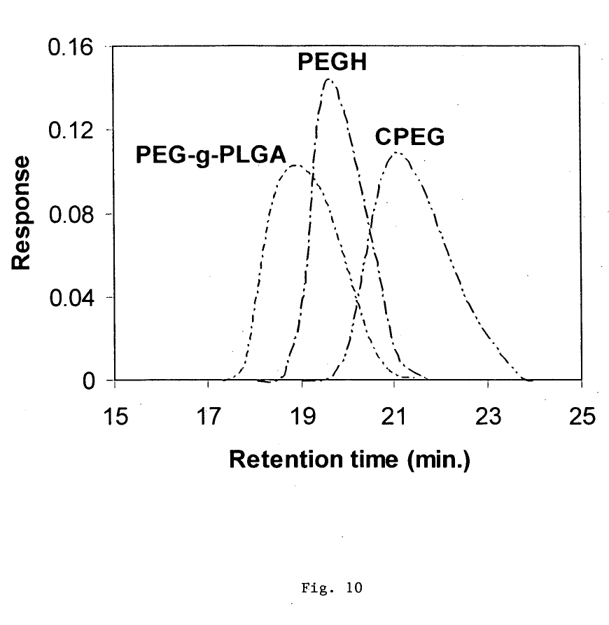

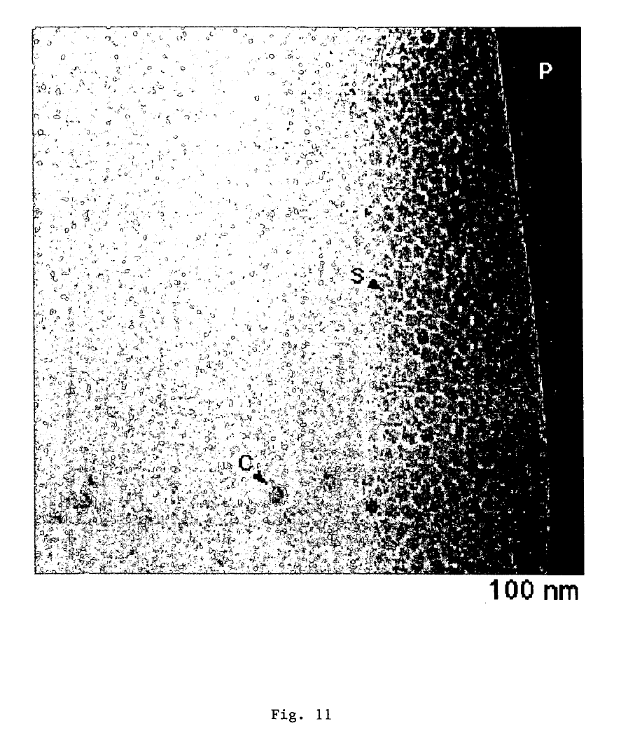

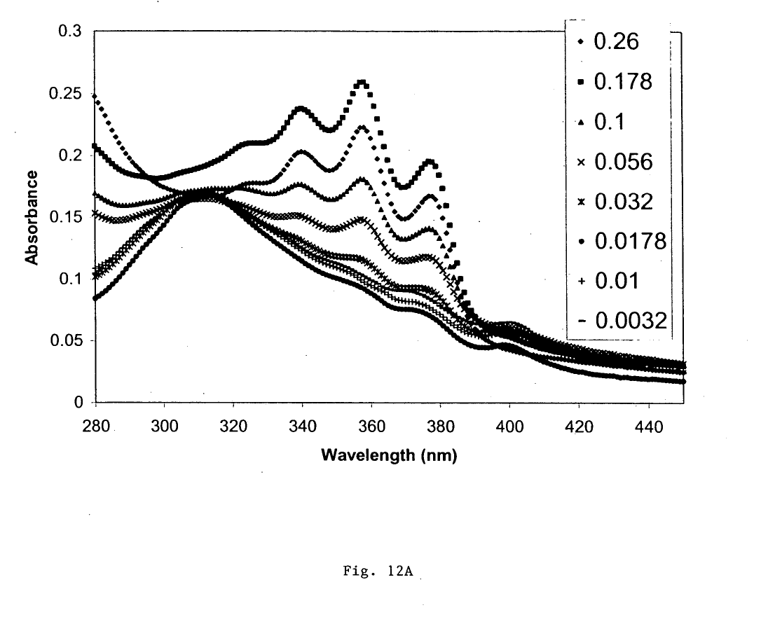

There are two possibilities of the ring-opening pattern of the epoxy group during the reaction of EPEG and CPEG. The nucleophiles prefer to attack the sterically less hindered side of the epoxy group in the base-catalyzed addition, while ring opening is less regiospecific in cationic polymerization. A GPC chromatogram (FIG. 10) showed the increase in molecular weight by the formation of PEGH from CPEG and EPEG. Assuming a PEGH molecular weight of about 3,000, there are ˜2-3 pendant hydroxy groups per each PEGH.

The resultant PEGH was used as an initiator for the ring-opening polymerization of DL-lactide and glycolide in the presence of stannous octoate as a catalyst. H-NMR spectra (FIG. 9) showed an ethylene glycol unit at 3.6 ppm, a lactic acid unit at 5.3 ppm (methine) and 1.8 ppm (methyl), and a glycolic acid unit at 4.8 ppm. Composition of the PEG-g-PLGA calculated by ¹H-NMR was 2.98/2.35/1.00 (ethylene glycol/DL-lactic acid/glycolic acid) in mole ratio. The methylene protons of the epoxy group show up at 2.6 ppm and 2.8 ppm in H-NMR. In the H-NMR spectrum of PEGH and PEG-g-PLGA the epoxy signals are too small to be Analyzed quantitatively. Weight average molecular weight (M_{w}) and PDI of PEG-g-PLGA determined by GPC relative to polystyrene standards was 11,000 and 1.3, respectively.

Gel Permeation Chromatography (GPC):

The GPC system (Waters 515) with a refractive Index Detector (Waters 410) and a Light Scattering Detector Mini Dawn (Wyatt Technology) were used to obtain molecular weight and molecular weight distribution. Styragel® HMW 6E and HR 4E columns (Waters) were used in series. Tetrahydrofuran (THF) was used as an eluting solvent.

Cryo-transmission Electron Microscope (Cryo-TEM):

Using cryo-TEM, a 1% (by weight) PEG-g-PLGA solution was investigated in the form of vitreous films. Detailed procedures for the sample preparation have been published elsewhere. Bellare et. al., Electron Microsc. Tech. 10:87-111 (1999). The liquid films of 10 to 300 nm thickness freely spanning across the micropores in a carbon-coated lacelike polymer substrate were prepared at about 23.7° C. with control of temperature and humidity, and rapidly vitrified with liquid ethane at its melting temperature (−180° C.). Imaging was performed using a JEOL 1210 operating at 120 kV. Adequate phase contrast was obtained at a nominal underfocus of ˜6 micrometers. Images were recorded on a Gatan 724 multiscan camera, and optical density gradients in the background were digitally corrected.

CMC Determination:

Hydrophobic dye, 1,6-diphenyl-1,3,5-hexatriene (DPH) was dissolved in methanol with a concentration of 0.4 mM. This solution (20 μL) was injected using a microsyringe into 2.0 ml PEG-PLGA polymer aqueous solution with various concentrations between 0.0032% and 0.26% by weight and equilibrated for 5 hours at 4° C. UV-VIS spectrometer (HP 8453) was used to get the UV-VIS spectra in the range of 280 to 450 nm at 20° C. CMC was determined by the plot of the difference in absorbance at 377 nm and at 391 nm (A₃₇₇−A₃₉₁) versus logarithmic concentration.

Viscosity:

The viscosity of PEG-g-PLGA aqueous solutions (22% by weight) was measured as a function of temperature. A Canon-Fenske viscometer 200 with a viscometer constant of 0.0966 centistokes/sec. was used to measure the viscosity of the polymer solution.

Dynamic Mechanical Analysis:

The solution-to-gel (sol-gel) transition of the graft copolymer aqueous solution (22% by weight) was investigated using a dynamic mechanical rheometer (Rheometric Scientific: SR 2000). The polymer solution was placed between parallel plates having a diameter of 25 mm and a gap distance of 0.5 mm. The data were collected under controlled stress (4.0 dyne/cm²) and frequency of 1.0 radian/second. The heating and cooling rate was 0.2° C./minute.

Sol-gel Transition:

The sol-gel transition was determined by a test tube inverting method with a temperature increment of 1° C. per step. Polymer aqueous solutions (0.5 g) were prepared in 4 mL vials with inner diameters of 11 mm. The vials were immersed in a water bath at each step for 15 minutes. The sol-gel transition temperature was monitored by inverting the vials, and if there was no flow in 30 seconds, it was regarded as a gel. The transition temperature was determined with ±1° C. accuracy.

NMR Study:

A NMR spectrometer (Variant VXR 300) was used for ¹H-NMR and ¹³C-NMR to study composition and microenvironment change during solution-to-gel (sol-to-gel or sol-gel) transition. For the ¹³C-NMR in D₂O, a 22% (by weight) PEG-g-PLGA solution was prepared.

Results and Discussion

Micellization:

PEG-g-PLGA is an amphiphilic copolymer and a core-shell structure can be expected in water. The hydrophobic PLGA side chains form a core and the hydrophilic PEG backbones form a shell region. The formation of core-shell structure was investigated by Cryo-transmission electron microscopy (Cryo-TEM) and dye solubilization method.

The formation of micelles was directly confirmed by a Cryo-TEM image. A 1% (by weight) PEG-g-PLGA solution at 23.7° C. was quenched into a vitrified form at −180° C. The Cryo-TEM image shows closely packed spherical micelles (denoted as S in FIG. 11) on the left side of black stripe. The diameter of a micelle is about 9 nm (FIG. 11).

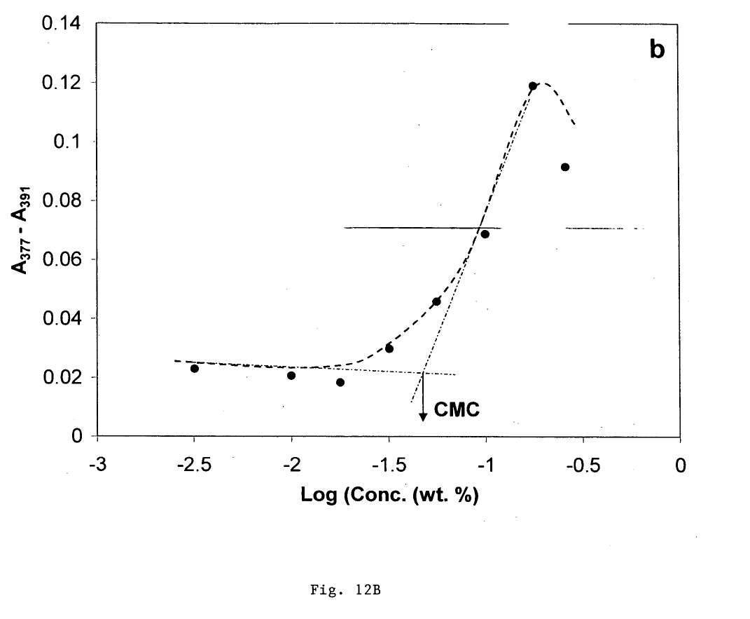

At a fixed concentration of DPH, the polymer concentration was increased from 0.0032 to 0.26% (by weight). The absorption coefficient of the hydrophobic dye (DPH) is much higher in a hydrophobic environment than in water. Thus, with increasing polymer concentration, the absorbance at 377 and 356 nm increased, indicating that the polymers formed a core-shell structure in water creating a hydrophobic environment (FIG. 12A). The critical micelle concentration (CMC) was determined by extrapolating the absorbance at 377 nm minus absorbance at 391 nm (A₃₇₇−A₃₉₁) versus logarithmic concentration (FIG. 12B) to compensate for the scattering effect. The CMC value determined by this extrapolation is not precise due to the uncertainty in the line, but it is in a range of 0.01-0.05% (by weight) at 20° C. FIG. 12A is a UV spectrum showing the formation of core-shell structure of polymers in water at 20° C. DPH concentration was fixed at 4μM and polymer concentration (% by weight) varied: 0.0032, 0.01, 0.0178, 0.032, 0.056, 0.010, 0.178, 0.26. The increase in absorption band at 377 nm with increasing polymer concentration indicates the formation of a hydrophobic environment, that is, micelles, in water. FIG. 12B shows a CMC determination by extrapolation of the difference in absorbance at 377 nn and at 391 nm.

Sol-gel Transition:

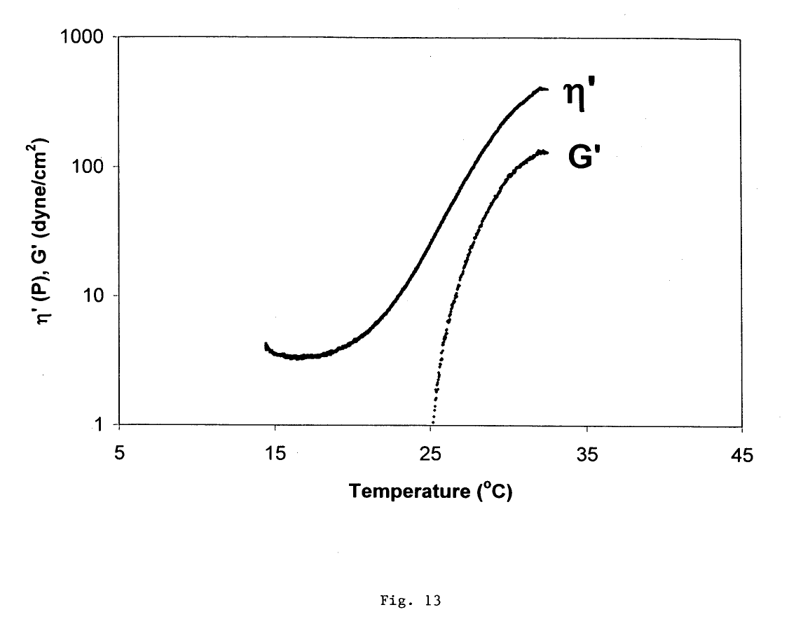

At high concentrations, the PEG-g-PLGA aqueous solution undergoes a sol-to-gel transition with increasing temperature. The viscosity of a 22% (by weight) PEG-g-PLGA aqueous solution that was measured by Cannon-Fenske viscometer was 27 centipoises at 20° C. This viscosity is low enough for an easy formulation of the polymer with pharmaceutical agents that could be injected using a 22-gauge needle. Above the gelation temperature, the viscosity is too high to flow through the capillary of this viscometer. Dynamic mechanical Analysis of 22% aqueous polymer solutions show that the real part (η′) of complex viscosity increases from 5 to 500 dyne sec cm⁻² [P] and elastic modulus (G′) increased from zero to 100 dyne cm⁻² during a sol-to-gel transition (FIG. 13). η′ and G′ are measures of dissipated energy and stored energy respectively when a material is subject to cyclic deformation. And, practically no flow was observed above 30° C. in the test-tube inverting method, indicating a sol-to-gel transition. When the two methods for 22% aqueous polymer solutions are compared, the gelation temperature determined by test-tube inverting method corresponds to the temperature at which η′ of 100 P and G′ of 50 dyne/cm² are reached in dynamic mechanical Analysis when thermal equilibrium is assumed in both cases.

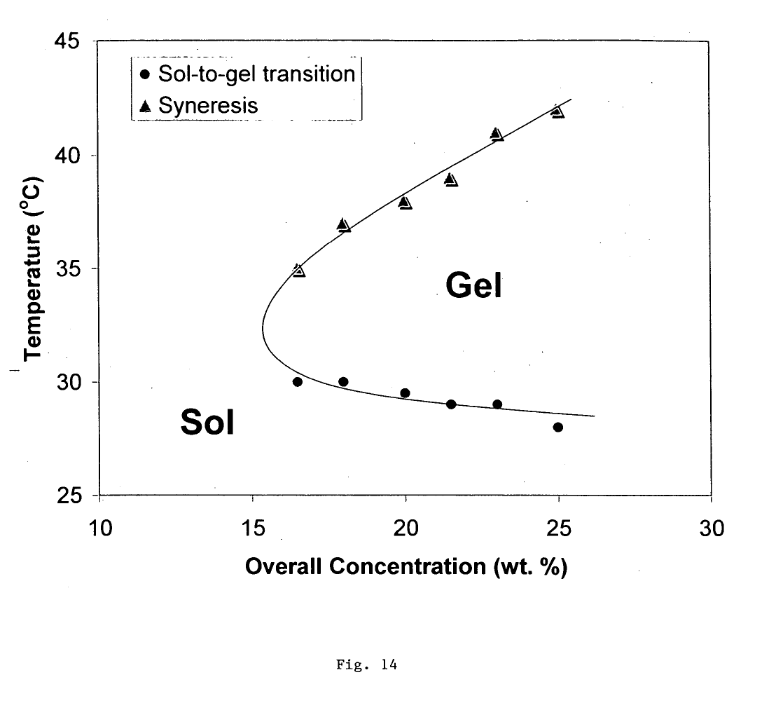

The phase diagram of PEG-g-PLGA aqueous solutions determined by a test-tube inverting method is shown in FIG. 14. The sol-to-gel transition is accompanied by a sharp increase in viscosity. The critical gel concentration (CGC) above which the gel phase appears was about 16%. Below CGC, the system flows even though the viscosity increases as the temperature increases. The sol-to-gel transition temperature, estimated at about 30° C., was slightly affected by the polymer solution concentration. Thus, such a sol-gel can be formulated at room temperature and would form a gel in situ upon subcutaneous or intramuscular injection. Pharmaceutical agents retained in the gel would slowly release from the in situ formed gel.

Further analysis of the phase diagram illustrates that with increasing temperature the gel exhibits syneresis, marked as gray triangles in FIG. 14, a macromolecular phase separation where some amount of water is exuded from the gel phase. Above the syneresis temperature, the gel phase remains separated from the water. Therefore, the sol phase at low temperature is a homogeneous one-phase solution while the sol phase above syneresis is a two-phase system. The gel region, right side of the trend line in the phase diagram indicates the area where a uniform gel phase exists. Based on the phase diagram shown in FIG. 14, 21-25% of PEG-g-PLGA aqueous solutions may be particularly useful injectable formulations for drug delivery.

The aggregation number of a micelle can be estimated from the size of the micelle by assuming that the micelle is a hard sphere. The radius of a micelle can be estimated from equation.

a. R=(3M_{s.D}.v₂/4ρN_{A})^{1/3}

Where M_{s.D}. denotes the molecular weight of a micelle obtained from centrifugal sedimentation, which is close to weight average molecular weight (M_{w}). v₂, and N_{A} are the partial specific volume of the polymer, and the Avogadro's number, respectively. The aggregation number of a micelle (N_{ag}) is given by equation.

b. N_{ag}=M/M₀

Where M and Mo denote molecular weight of a micelle and molecular weight of a polymer respectively. Assuming v₂ is 0.95, which is typical for polyester or polyether, and R is about 4.5 nm (diameter ˜9 nm) from Cryo-TEM, the micellar aggregation number is 40 at 20° C. The aggregation number of a micelle is assumed to be practically constant for a sol region as in the cases of PEG-PLGA-PEG and poloxamer 407. This calculation also assumes that M is equal to M_{s.D} and the molecular weight of PEG-g-PLGA (M₀) is 6000 as determined from GPC data. Based on this estimation, the thermodynamic functions such as enthalpy (ΔH⁰), Gibbs free energy (ΔG⁰), and entropy of gelation (ΔS⁰) can be calculated. Now, the standard states of gelation process are taken to be the micelles in ideal dilute solution at unit molarity and micelles in gel state.

c. ΔG⁰=RT_{gel}1n Cₘ

d. ΔH⁰=R[d 1n Cₘ/d(1/T_{gel})]

e. ΔS⁰=(ΔG⁰−ΔH⁰)/T_{gel}

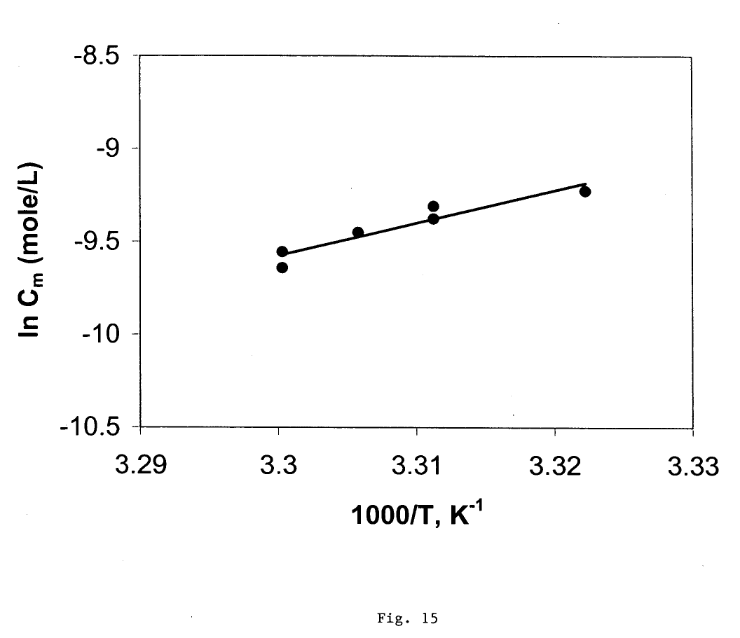

Cₘ is the concentration of micelles in mole L⁻¹ that is calculated by assuming that the aggregation number per micelle is 40. T_{gel} is the sol-to-gel transition temperature. ΔH⁰ calculated from the slope of In Cₘ versus 1/T_{gel} (FIG. 15) is 146 kJ mole⁻¹ (micelle) or ΔH⁰=3.65 kJ mole⁻¹ (chain). This value is similar to gelation of poloxamer 407 (ΔH⁰=1.5 kJmole⁻¹ (chain)) and PEG-PLGA-PEG triblock copolymers (ΔH⁰=1.32 kJ moles⁻¹ (chain)). Gibbs free energy (ΔG⁰) and entropy (ΔS⁰) for the gelation of 22 wt. % PEG-g-PLGA aqueous solution with a T_{gel} of 30° C. are −0.59 kJ mole⁻¹ and 1.9 J mole⁻¹ K⁻¹, respectively. This calculation leads to the conclusion that the entropy drives the gelation. The molecular origin of such an entropy-driven process has been suggested as hydrophobic interactions. Water molecules tend to surround the hydrophobic segment (PLGA) to decrease the free energy. Consequently, the entropy of water molecules decreases in the presence of hydrophobes. Therefore, the surface area of hydrophobic molecules is minimized in water. Such hydrophobic interactions increase with increasing temperature, and change the molecular conformation of PEG-g-PLGA. This might drive the gelation.

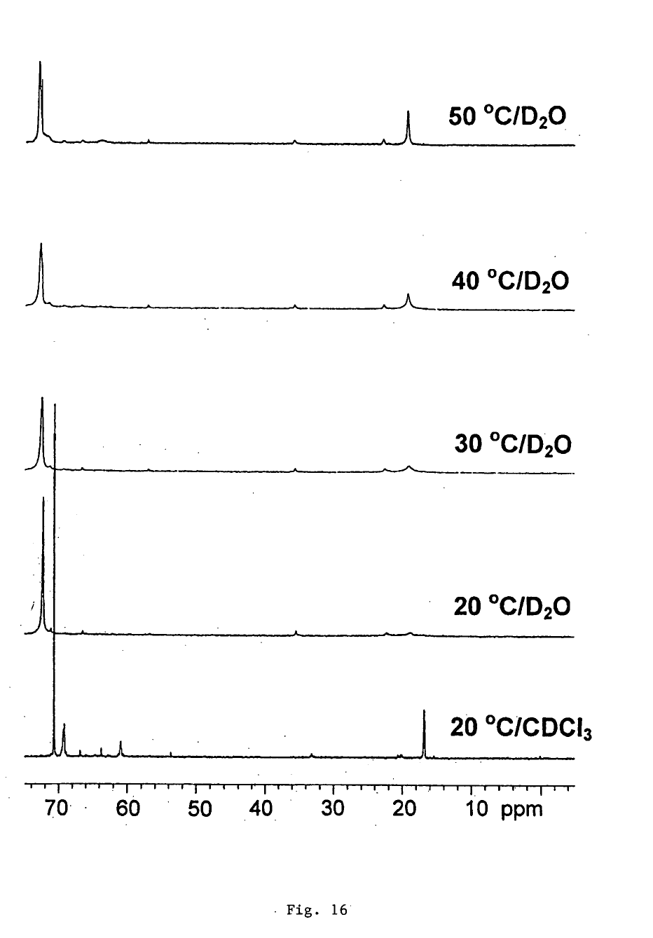

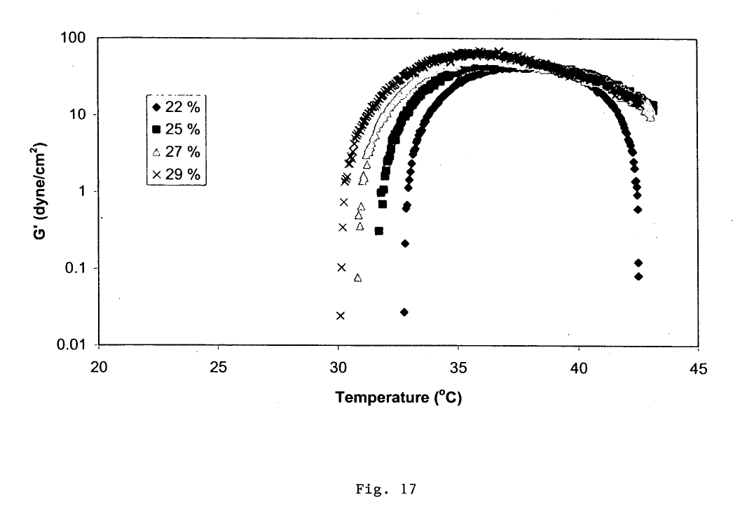

The ¹³C-NMR Analysis of the polymers was conducted at different temperatures to elucidate the structure of the gel and mechanism of gel formation (FIG. 16). Spectra of polymers dissolved in water and chloroform were compared. The ¹³C-NMR spectra of a 22% (by weight) PEG-g-PLGA in D₂O were obtained at about 20° C. (sol state), about 30° C. (just above sol-to-gel transition), about 40° C. (gel state), and about 50° C. (macrophase-separated state) by simply increasing the temperature around the probe without changing NMR parameters. The equilibration time at each temperature was 15 minutes. Chloroform (CDCl₃) is a nonselective good solvent for both PEG and PLGA blocks while water (D₂O) is a good solvent for PEG but is a poor solvent for PLGA. The sharp peaks of both PEG and PLGA in chloroform are compared with a collapsed peak of PLGA in water at first and second rows of ¹³C-NMR, indicating core (PLGA)-shell (PEG) structure of the polymer in water. The molecular motion of PEG in water is decreased due to anchoring effects by the hydrophobic PLGA segments compared with that in chloroform. This is reflected in a broadened peak of PEG in D₂O at 20° C. The change in molecular association at sol-to-gel transition involves the change in molecular motion of the polymers. The change in ¹³C-NMR with increasing temperature (20° C.-50° C.) shows such a change in microenvironment around the PEG and PLGA. The PEG peak (72 ppm) at a gel state (30° C.) is broadened and decreased by half in height compared with a sol state (20° C.), whereas there is a slight increase in PLGA peak height (20 ppm) at gel state (30° C.). These changes in peak heights indicate a significant decrease in molecular motion of the PEG backbone, and increased thermal motions of the PLGA side chains during sol-to-gel transition. This behavior is quite different from that of PLGA-g-PEG. PLGA-g-PEG showed little change in PEG peak during sol-to-gel transition at the ¹³C-NMR in D₂O. Based on these observations, the following model can be suggested for the sol-to-gel transition of PEG-g-PLGA copolymer aqueous solutions. In a sol state, the polymer conformation is micellar where the PEGs occupy shell and PLGAs occupy core of the micelle. The degree of association in a sol state is not enough to form a three dimensional network. With increasing temperature, the hydrophobic interactions increase and associations of polymers decrease the PEG molecular motion, resulting in a long-range network formation. The degree of association is strong enough to keep its integrity in the presence of excess water at a given temperature such as about 37° C. Therefore, this system is a gel rather than a solution with an increased viscosity. As the temperature increases further, the long-range interactions among the polymers increase and phase mixing between PEG and PLGA occurs, resulting in the macrophase separation between water and polymer that occurs at about 50° C.

The 22% polymer solutions (0.5 g) are injected into 4 mL vials (diameter of 1.1 cm) and kept in a 37° C. water bath for five minutes. During this time the gel forms. 3 mL of phosphate buffer saline (37° C., pH=7.4) is added and the vials are shaken (16 strokes/minute) in the water bath to simulate body condition. The gel keeps its integrity for one-week in vitro, and the initially turbid gel becomes transparent in 3 to 7 days. After 7 days, the gel totally disintegrated to become a clear polymer solution.

This material can be applied for a short-term delivery of bioactive agents such as pharmaceutical drugs (e.g. proteins, anticancer drugs) as well as a carrier or delivery system for bioactive agents used in tissue engineering. The hydrophobicity of the drug and the molecular structure of the polymers affect the extent of diffusion or degradation dominant drug release profile. Therefore, by choosing the appropriate drug and molecular parameters of PEG-g-PLGA, a short-term delivery system can be designed based on this polymer hydrogel.

### EXAMPLE 8

This is an example of a one-step PLGA-g-PEG synthesis. The graft copolymer PLGA-g-PEG was synthesized by a one-step ring opening polymerization of DL-lactide, glycolide, and epoxy terminated poly(ethylene glycol) (PEG; m.w.=600) using stannous octoate as a catalyst. The DL-lactic acid/glycolic acid/ethylene glycol mole ratio is 3.2:1:2.8, which was determined by H-NMR. Therefore, the grafting frequency of PEG is 4.7% by mole. Gel permeation chromatography (GPC) using light scattering and refractive index detectors in series can give absolute molecular weight of polymers. P. J. Wyatte, 1 Annal. Chim. Acta, 272 (1993). GPC shows a unimodal curve. The number average molecular weight (Mn) and polydispersity (Mw/Mn) of the polymers determined by GPC using tetrahydrofuran (THF) as an eluting solvent are 9300 and 1.5, respectively. Therefore, the 4˜5 PEGs are grafted on a PLGA backbone.

Sol-gel Transition:

At room temperature, viscosity of the 25% (by weight) PLGA-g-PEG aqueous solution is about 0.3 poise (gm⁻¹s⁻¹), which allows for injecting the solution using a 25-gauge needle. With increasing temperature, the aqueous solutions of 25% PLGA-g-PEG undergo a sol-to-gel transition at 30° C. Further increase in temperature of the

PLGA-g-PEG aqueous solution results in a macroscopic phase-separation between gel and water, that is, syneresis, which occurs at about 50° C. in this example.

Dynamic Mechanical Analysis:

The sol-gel transition of the graft copolymer aqueous solution was investigated using dynamic rheometry (Rheometric Scientific: SR 2000) in a similar manner to poloxamer aqueous solutions. The polymer solution was placed between parallel plates having a diameter of 25 mm and a gap distance of 0.5 mm. The data were collected under controlled stress (4.0 dyne/cm²) and a frequency of 1.0 radian/second. The heating and cooling rate was 0.2° C./minute. By the dynamic mechanical Analysis, sol-gel transition can be identified in a more reproducible and quantitative manner than the test-tube inversion method.

The modulus of the PLGA-g-PEG aqueous solution is shown in FIG. 16 as a function of temperature and concentration. The storage modulus increases abruptly at the sol-to gel transition. The gels have a modulus of about 50 dyne/cm² and are slightly affected by concentration in a range of 22% to 29% (by weight). The sol-to-gel transitions occur at around 30° C., allowing formulation at room temperature.

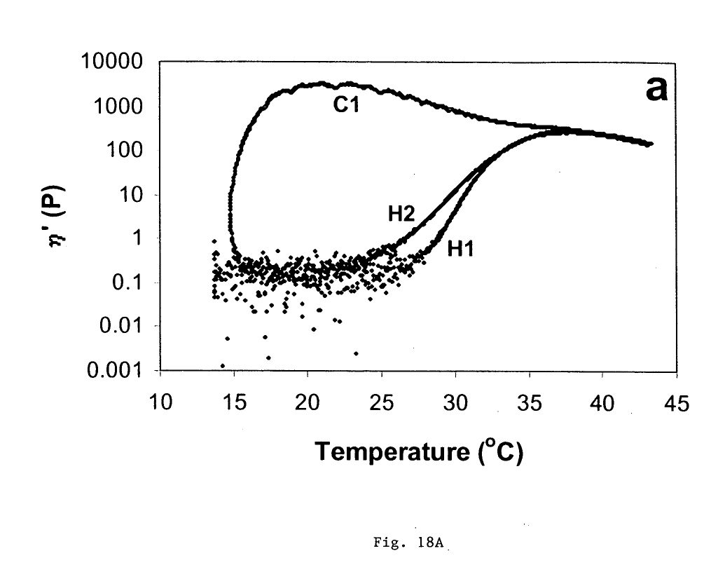

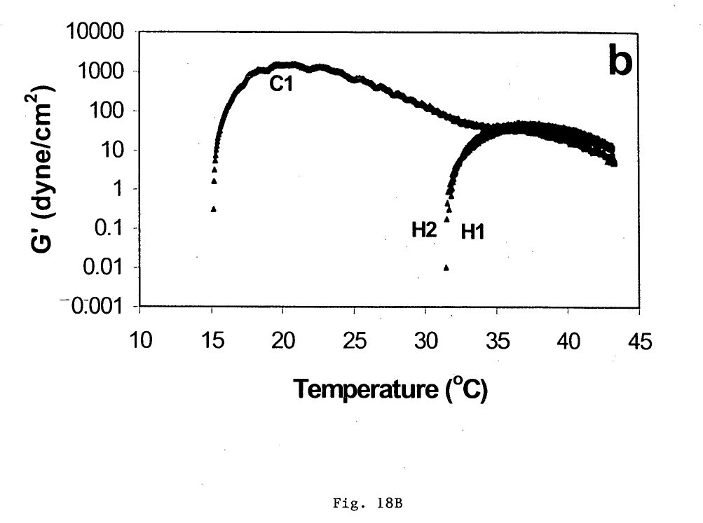

To confirm the reversibility of the sol-gel transition, 25% PLGA-g-PEG aqueous solutions were studied. The real part (η′) of complex viscosity of the polymer 20 solution, which is a measure of dissipated energy when cyclic deformation is applied to a material, is shown as a function of temperature in FIG. 18A. During the first heating cycle (H1), η′ increased 1000 times upon sol-to-gel transition. The cooling curve (C1) shows that the gel phase persisted over the temperature range of 43˜20° C. in the experimental time scale. This fact results from the difficulty in molecular rearrangement 25 in the gel phase; once the solution forms a gel, the molecules resist disintegration. η′ abruptly decreased at about 15° C. due to gel-to-sol transition during the cooling of the system. The second heating curve (H2) shows sol-to-gel transition at practically the same temperature as the first heating curve (H1).

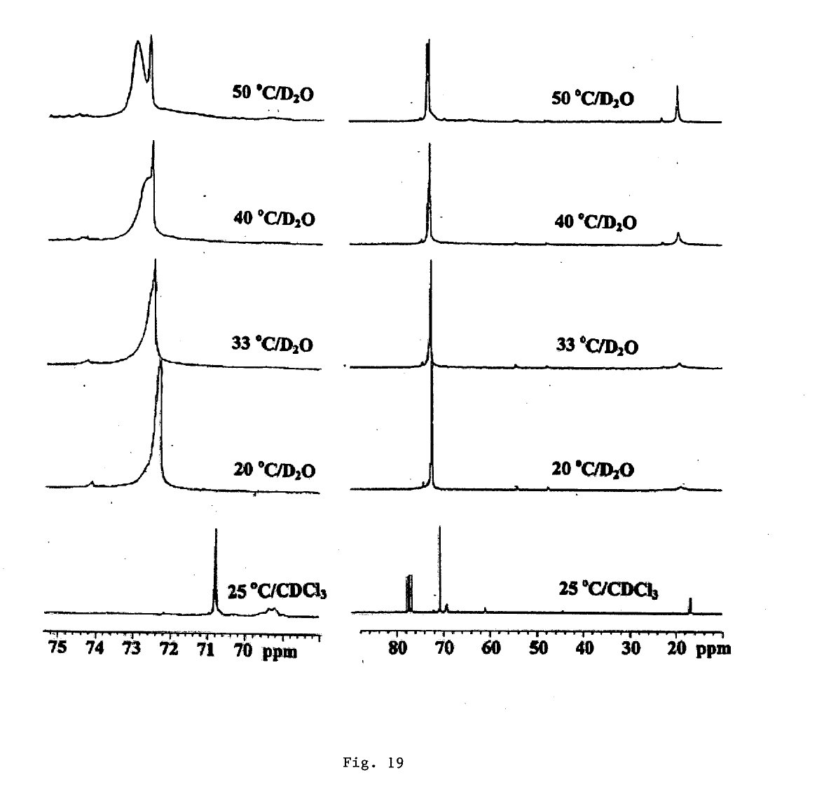

The storage moduli (G′) of the PLGA-g-PEG aqueous solutions (25% by weight), which are a measure of stored energy when a cyclic deformation is applied to a material, are practically zero at a sol state and are not shown in the heating curve (FIG. 18B; H1). G′ sharply increased during sol-to-gel transition at 32° C. as shown in heating curves. The maximum value for G′ was measured between 35˜39° C., indicating a promising material for in-vivo applications. During the cooling cycle (C1), the gel modulus increased over the temperature range of 43˜20° C., exhibiting similar behavior to typical elastic materials, and dropped abruptly at about 15° C. due to gel-to-sol transition. During the first (H1) and second (H2) heating cycle, practically the same transition curve was measured for G′, indicating a reversible gelation. The decrease in G′ at temperatures above 40˜45° C. can be expected due to increase in thermal motion. This trend also was observed with ¹³C-NMR spectra (FIG. 19).

NMR Study:

The ¹³C-NMR spectra (FIG. 19) of a 25% copolymer solution in D₂O were recorded at different temperatures. In the sol state (20° C.), the methyl peak of the hydrophobic PLGA (18 ppm) is collapsed and broadened compared with PEG peak (72 ppm) whereas that in CDCl₃ appears as a sharp peak, indicating core-shell structure of this polymer in water. The core-shell structure of these amphiphilic copolymers was also confirmed by micelle formation in diluted aqueous solutions. The critical micelle concentration (CMC) determined by a dye solubilization method was 0.03% at 20° C.

Just above the sol-to-gel transition temperature (about 33° C.) of an aqueous 25% PLGA-g-PEG copolymer solution, the ¹³C-NMR peak shapes of both the hydrophobic PLGA methyl peak and hydrophilic PEG peak are preserved except that the PEG peak was shifted down field about 0.3 ppm. With a further increase in temperature, the peak height of the PLGA methyl peak increases, and the PEG peak is split into two peaks, a sharp one at 72.4 ppm and a broad one at 72.7 ppm. These behaviors are thought to be caused by an increase in molecular motion of the hydrophobic backbone and phase mixing between PEG and PLGA. The phase mixing between PEG and PLGA or PLLA was previously reported. Further increase in temperature resulted in macrophase separation between water and the polymer.

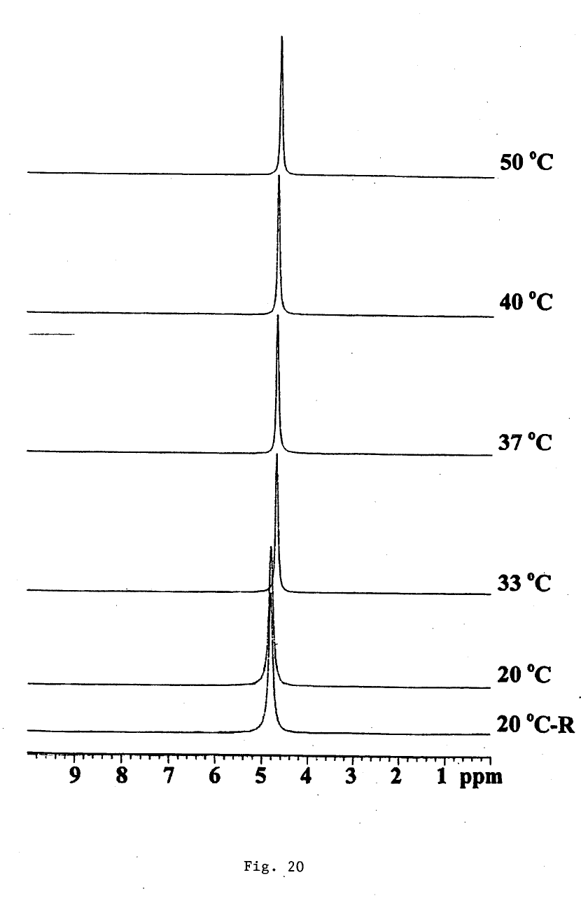

The reversibility of the sol-gel transition is also confirmed by deuterium NMR (FIG. 20). The peak at 4.8 ppm at 20° C. (sol state) shifted to 4.6 ppm at 33° C. (just above sol-to-gel transition), 4.58 ppm at 37° C. (gel state), 4.56 ppm at 40° C., and 4.5 ppm at 50° C. (syneresis). The change in chemical shift was the most pronounced during the sol-to-gel transition (δ=0.2 ppm) and then during syneresis. When the system is cooled to 20° C., the deuterium peak reappears at 4.8 ppm, indicating the reversibility of the transition. In a sol state, water moves more freely than in a gel state. During the sol-to-gel transition, PEG becomes more hydrophobic due to dehydration and the extent of hydrogen bonding between water molecules and polymers changes. Therefore, the time average environment around deuterium nuclei will be affected, leading to the changes in chemical shift of water during sol-to-gel transition. This finding suggests that the deuterium NMR can be used to determine sol-gel transition.

The sol-to-gel transition temperature can be controlled from 200 to 40° C. by changing PEG length and composition. When the PEG molecular weight of PLGA-g-PEG increases from 600 to 1000 the sol-to-gel transition occurred at 40° C., whereas the sol-to-gel transition occurred at 20° C. when the PEG composition is decreased by 20% by mole.

Varying Gel Durability:

The time frame for gel durability can be varied by adjusting the ratio of the two blocks in the formula Aₙ(B) (where n is >2). To test and exemplify the duration of a gel, 0.5 g of a polymer solution (see table below) was injected into a 4 ml vial (inner diameter 1.1 cm) and maintaining at about 37° C. for 5 minutes to allow the gel to form. After the gel is formed, 3 ml of phosphate buffer saline (37° C., pH=7.4) is added and the vial is placed in a shaker bath (16 strokes/minute). The polymer was monitored daily for complete degradation and the top layer of the buffer was replaced at the time of monitoring. Table 2 shows resulting gel durations for various ratios of the polymer.

PEG-g-PLGA had a molecular weight of about 11,000 and PLGA-g-PEG had a molecule weight of about 7,800.

TABLE 2
Gel durations for different polymer ratios.

| | Ratio (PEG-g-PLGA/PLGA-g-PEG) | | | | |
|---|---|---|---|---|---|
| | 100/0 | 60/40 | 50/50 | 40/60 | 0/100 |
| Duration of a gel | 1 day˜1 | 3 days˜2 | 2˜4 | 3˜6 | 6˜10 |
| measured as described | week | weeks | weeks | weeks | weeks |
| above. | | | | | |

Conclusions

The aqueous solutions of PEG-g-PLGA copolymers exhibited sol-to-gel transition in response to an increase in temperature. Micelle formation was confirmed by Cryo-TEM and dye solubilization method. The micellar diameter was about 9 nm and CMC was in a range of 0.01-0.05% by weight. ¹³C-NMR shows that the molecular motion of PEG backbones decreases while that of PLGA side chains increases during sol-to-gel transition.

The 21-25% PEG-g-PLGA solutions exhibit low viscosity at room temperature and form gels at body temperature. The gel morphology changed from turbid to transparent, and the integrity of gel persisted for one week.

The aqueous PLGA-g-PEG system showing a reversible sol-to-gel transition by increasing temperature was studied by dynamic mechanical Analysis and NMR 20 spectrophotometer. The rheological study of the copolymers in aqueous solution demonstrated that thermogelation occurred at about 30° C. and the elastic gel modulus exhibited a maximum around body temperature (about 37° C.). A preliminary in vivo study in a rat model confirmed in situ gel formation after subcutaneous injection of a 0.5 ml aqueous solution. The gel was still present at the injection site after 2 months.

Thermosensitivity enables the in-situ gel formation upon injection, therefore no surgical procedure is required to implant the drug delivery system and no organic solvent is needed for drug formulation. The physical properties of soft hydrogels reduce mechanical tissue irritation surrounding the injection site. Also, the polymers are biodegradable; therefore there is no need for surgical removal of the implant after the release of the pharmaceutical agent.

In specific embodiments where the therapeutic agent carrier includes the above-described thermogelling, biodegradable polymers, the therapeutic agent includes a radioactive therapeutic agent, such as a colloid including radioactive insoluble salt particles, for example, ⁹⁰YPO₄ particles. In still more specific examples, such a therapeutic agent carrier includes fibrin.

In other embodiments the stimulus sensitive gel is an enzymatically degradable polypeptide gel. Such gels are disclosed in co-pending U.S. patent application Ser. No. 10/124,614, which is herein incorporated by reference. The peptide bonds in the polypeptide carriers are more stable against hydrolysis than are the ester bonds in PEG/PLGA polymer carriers, thereby providing superior storage stability. Thus, the polypeptide carriers can be stored as water solutions, requiring no reconstitution.

In some cases the polypeptide gels include a biodegradable polymer having a biodegradable polypeptide block linked to a second polymer block to form a graft or linear polymer. The polypeptide gel satisfies a general structure comprising the formula of CₙDₘ, wherein n is equal to or greater than 1, m is equal to or greater than 1, C is a biodegradable polypeptide block, and D may include a biocompatible, soluble polymer having a length such that if D is not biodegradable, D may be eliminated by the kidney through the glomeruli filtration system. When D includes a biodegradable polymer, the length of D is not so limited. For example, D may include the same polypeptide block polymer as C, a different polypeptide block polymer or a non-polypeptide polymer, such as PEG. D may include any structure that would allow modification to accommodate active groups such that the polypeptide block may be coupled thereto.

The thermogelling, biodegradable, aqueous polymers, when prepared according to the formula CₙDₘ, work well with n and/or m between 1 and 20. In some cases the polypeptide block has an average molecular weight of from about 300 to about 30,000, and in certain more specific cases, from about 500 to about 10,000.

Polypeptides suitable for the polypeptide carrier in some cases include two or more amino acids wherein at least one of the amino acids is hydrophilic or hydrophobic as compared to the other amino acid(s) of the polypeptide. That is, there is a hydrophilic/hydrophobic relationship between the amino acids of the polypeptide. The polypeptide constituents can include alanine (A), isoleucine (I), phenylalanine (F), threonine (T), arginine (R), asparagine (N), aspartic acid (D), cysteine (C), glutamine (Q), glutamic acid (E), glycine (G), histidine (H), leucine (L), lysine (K), methionine (M), proline (P), serine (S), tryptophan (W), tyrosine (Y) or valine (V). The resulting polypeptide can include any combination of the listed constituents having a hydrophilic/hydrophobic relationship therebetween.

For example, the polypeptide carrier can include a polypeptide/polyethylene glycol copolymer. In one example of a polypeptide-polyethylene glycol (PP/PEG) copolymer system, the polypeptide block has an average molecular weight of from about 300 to about 30,000, and more preferably from about 500 to about 10,000. It is preferred that the polyethylene glycol (PEG) block have an average molecular weight of between about 300 and 20,000 and more preferably between about 500 and 10,000. The PEG block with a higher molecular weight than 10,000 may be difficult to filter through glomeruli filtration.

Although U.S. patent application Ser. No. 10/124,614, contains a detailed description of multiple syntheses for the disclosed therapeutic agent carriers including enzymatically degradable polypeptide polymers, syntheses of specific examples of such polymers are discussed below for convenience.

### EXAMPLE 9

In this example polypeptide/polyethylene glycol polymers are synthesized.

Materials:

Methoxy terminated polyethylene glycol (m.w. from about 300 to about 30,000; Aldrich), polyethylene glycol (m.w. 1000; Aldrich), hexamethylene diisocyanate (HMDI), N-carboxy anhydride-Isoleucine (NCA-Isoleucine), NCA-phenylalanine may be synthesized using standard methods as known to those persons skilled in the art.

General Synthesis:

N-carboxy anhydride (NCA) of alpha-amino acid, such as alanine (A), isoleucine (I), phenylalanine (F), threonine (T) are synthesized by reacting alpha amino acids and phosgene.

Triblock copolymers, polypeptide-b-poly(ethylene glycol)-b-polypeptide (PP-PEG-PP) are synthesized by combining polyethylene glycol with one or two kinds of NCA-alpha amino acids in toluene at a temperature of from about 20 to 120° C. A specific example is set forth below as illustration.

Poly(ethylene glycol)-b-polypeptide-b-poly(ethylene glycol) (PEG-PP-PEG) may be synthesized by combining methoxy terminated PEG with NCA-alpha amino acid to make a di-block copolymer of PEG-PP. The di-block copolymers are coupled using coupling agents such as hexamethylene diisocyanate (HMDI). Specific examples are set forth below for illustration.

Synthesis of Polyisoleucine-b-poly(ethylene glycol)-b-polyisoleucine (PI-PEG-PI):

About 0.5 to about 2 grams of NCA-isoleucine and about 1 gram PEG (m.w. about 1000) are mixed and reacted at about 60° C. for about 24 hours. Ethyl ether is added to the reaction mixture to precipitate out the resulting polyisoleucine-b-poly(ethylene glycol)-b-polyisoleucine (PI-PEG-PI). The product is placed under high vacuum (about 10⁻³ mm Hg) for about 48 hours to remove any residual solvent.

The sol-to-gel transition of the polyisoleucine-b-poly(ethylene glycol)-b-polyisoleucine (PI-PEG-PI) may be measured utilizing standard methods, such as the inverted test tube method or falling ball method, and is expected to be in the range of from about 0 to about 40° C. The molecular weight of the resulting polyisoleucine-b-poly(ethylene glycol)-b-polyisoleucine is expected to be from about 1500 to about 20,000.

Synthesis of Poly(isoleucine-co-phenyl alanine)-b-poly(ethylene glycol)-b-poly(isoleucine-co-phenylalanine) (PIF-PEG-PIF):

About 0.5 to about 1 gram of NCA-Isoleucine, from about 0.1 to about 1 g of NCA-phenylalanine, and about 1 gram of PEG (m.w. about 1000) are mixed and reacted at about 60° C. for about 24 hours. Ethyl ether is added to the reaction mixture to precipitate out resulting poly(isoleucine-co-phenyl alanine)-b-poly(ethylene glycol)-b-poly(isoleucine-co-phenylalanine) (PIF-PEG-PIF). The product is placed under high vacuum (about 10⁻³ mm Hg) for about 48 hours to remove any residual solvent.

The sol-to-gel transition of the poly(isoleucine-co-phenyl alanine)-b-poly(ethylene glycol)-b-poly(isoleucine-co-phenylalanine) is tested and expected to be in the range of from about 0 to about 40° C. The molecular weight is expected to be about 1500 to about 20,000.

Synthesis of Poly(ethylene glycol)-b-polyisoleucine-b-poly(ethylene glycol) (PEG-PI-PEG):

Poly(ethylene glycol)-b-polyisoleucine-b-poly(ethylene glycol) (PEG-PI-PEG) is synthesized by combining about 1 gram of methoxy terminated PEG (m.w. about 550) with from about 0.5 to about 2 grams NCA-isoleucine in toluene at about 60° C., resulting in a diblock copolymer of PEG-PP. The diblock copolymer of PEG-PP is coupled using hexamethylene diisocyanate (HMDI). Ethyl ether is then used to precipitate out the triblock copolymer, PEG-PI-PEG. The product is placed under high vacuum (about 10⁻³ mm Hg) for about 48 hours to remove any residual solvent.

The sol-to-gel transition of the PEG-PI-PEG is expected to be at about 0 to about 40° C. The molecular weight is expected to be from about 1500 to about 20,000.

Synthesis of Poly(ethylene glycol)-b-poly(isoleucine-co-phenyl alanine)-b-poly(ethylene glycol) (PEG-PIF-PEG):

Poly(ethylene glycol)-b-poly(isoleucine-co-phenyl alanine)-b-poly(ethylene glycol) (PEG-PIF-PEG) is synthesized by combining about 1 gram of methoxy terminated PEG (m.w. about 550) with about 0.5 to about 2 grams of NCA-isoleucine and about 0.1 to about 1 gram of NCA-phenylalanine in toluene at about 60° C. to form a diblock copolymer of PEG-PP. The PEG-PP diblock copolymer is coupled by using hexamethylene diisocyanate (HMDI). Ethyl ether is used to precipitate out the resulting triblock copolymer PEG-PIF-PEG.

The sol-to-gel transition of the resulting PEG-PIF-PEG is expected to be from about 0° to about 40° C. The molecular weight is expected to be from about 1500 to about 20,000.

Graft copolymers or other topologies having graft numbers greater than 2 are synthesized in similar manners, as would be readily achieved by those skilled in the art having reviewed this disclosure.

In specific embodiments where the therapeutic agent carrier includes the above-described biodegradable polymer having a biodegradable polypeptide block linked to a second polymer block to form a graft or linear polymer, the therapeutic agent includes a radioactive therapeutic agent, such as a colloid including radioactive insoluble salt particles, for example, ⁹⁰YPO₄ particles. In still more specific examples, such a therapeutic agent carrier includes fibrin.

In some examples one or more contrast or imaging agents are added to the therapeutic agent carrier. For nuclear medicine imaging, any gamma-emitting radioisotope can be added to the carrier as an imaging agent or contrast agent. The common types of gamma source imaging systems are gamma-cameras (Anger cameras), single-photon emission computed tomography (SPECT), and positron-emission tomography (PET). Preferred imaging agents for gamma cameras would be technetium-99m (and any of the standard chemical forms of Tc-99m, such as pertechnetate). The preferred chemical form of Tc-99m may be an insoluble material, such as Tc-99m-sulfur colloid (a common liver scanning agent). Other gamma emitters include but are not limited to indium-111, rhenium-186, rhenium-188, thallium-201, gallium-67, yttrium-91, and iodine-131, and combinations thereof. Positron-emission tomography systems use positron-emitting radioisotopes and detect the twin 0.511 keV photons that accompany radioactive decay. Examples of radioisotopes that could be added for positron-emission tomography include fluorine-18, copper-64, arsenic-74, and zirconium-89, ioding-124, and yttrium-86. A typical amount of photon-emitter added is one that will provide approximately 500,000 counts per two-minute imaging time (0.3 to 10 millicuries).

For ultrasonic imaging, any ultrasound contrast-enhancement agent can be added to render the therapeutic agent carrier more imageable using ultrasonic detection. Examples include commercially available echocontrast products such as Albunex® (registered trade mark, Molecular Biosystems) and Optison™ (trade mark of Molecular Biosystems), which are manufactured for and distributed by Mallinckrodt Medical, St. Louis, Mo. Albunex® is an ultrasound contrast agent prepared by sonicating 5% human serum albumin to produce stable, air-filled, albumin-coated microspheres. It is an effective ultrasound contrast agent for use during echocardiography and other ultrasound radiological procedures. Optison™ is an ultrasound contrast agent containing human serum albumin with octofluoropropane. Each milliliter of echocontrast agent contains about 700 million microspheres. The amount of contrast agent added could be approximately 1 to 5 percent by weight of the therapeutic agent carrier.

For magnetic resonance imaging (MRI), any paramagnetic material used for contrast-enhancement may be used. An example includes a relaxation agent gadolinium-chelate (gadolinium-DTPA or gadolinium-EDTA). The stable (nonradioactive) form of gadolinium is preferred. The amount of gadolinium contrast agent added would be a few parts per thousand by weight (millimolar concentrations).

The therapeutic agent carriers disclosed above are useful for the treatment, prevention, and/or inhibition of many diseases and conditions. For example, in some cases the disclosed therapeutic agent carriers are used as radioisotope carriers for infusional brachytherapy. In other cases the disclosed therapeutic agent carriers are used as anti-bacterial carriers to treat or prevent infections introduced during surgical operations. In still other instances the disclosed therapeutic agent carriers are used to carry anti-cancer agents, such as radiotherapeutic agents or chemotherapeutic agents in the treatment of cancer.

For example, in some cases, the therapeutic agent carrier is used to deliver a therapeutic agent to the margins of tissue after tumor resection. When a cancerous tumor is surgically resected from a subject (i.e., an animal, such as a mammal, for example, a human) the surgeon attempts to remove tissue beyond the location of the tumor. This is an imprecise art and may result in cancerous tissue being left in the subject's body at the margins of the removal site from which the tumor and tissue were resected. The disclosed therapeutic agent carrier compositions can be applied to the margins of the tumor removal site with anti-cancer agents designed to destroy vestigial cancerous cells, for example a ⁹⁰YPO₄ colloid. In other cases, the therapeutic agent carrier is injected into or around a tumor in vivo.

An amount of anti-cancer agent is used that is effective to destroy vestigial cancerous cells. One of ordinary skill in the art could determine this effective amount based on such considerations as the type of cancer, the suspected amount of vestigial cells, and the cancer cell killing properties of the anti-cancer agent. Guidance concerning examples of effective doses of 90-yttrium in killing certain types of cancerous cells is provided in certain examples in this specification, such as in Example 12 below.

Below are additional examples of certain disclosed stimulus sensitive gels and therapeutic agent carriers.

### EXAMPLE 10

An experiment was conducted to demonstrate the behavior of the gel of Example 5 during tissue perfusion in lymph nodes. A freeze-dried copolymer of N-isopropylacrylamide with acrylic acid (2 mole %) NiPAAm/AAc)] was dissolved in PBS as in Example 5. A dye Naphthol blue-black, electrophoresis reagent, from Sigma was added to the copolymer solution. In all solutions, the dye was physically mixed by dissolving into the solutions, but was not covalently bonded to the copolymer.

Canine lymph nodes were freshly isolated and equilibrated at 37° C. PBS for 30 minutes.

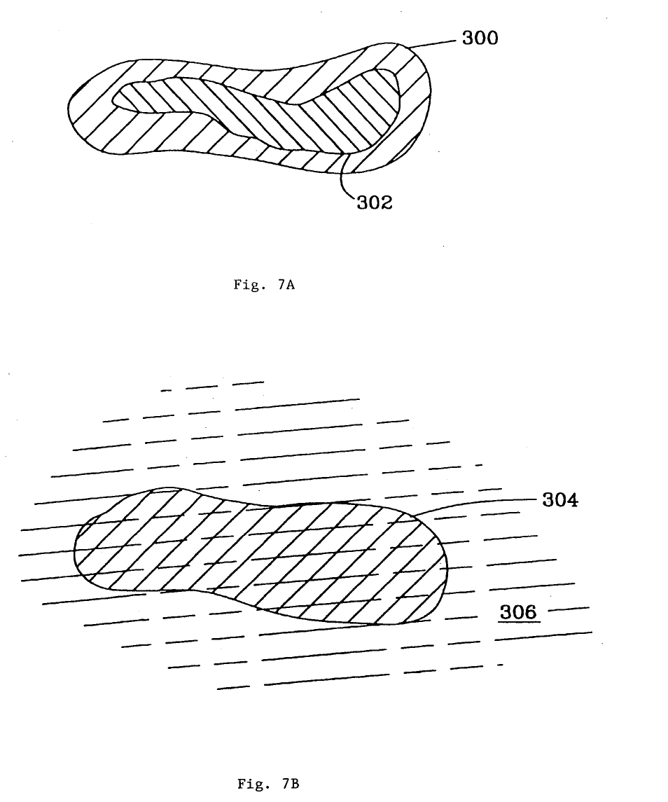

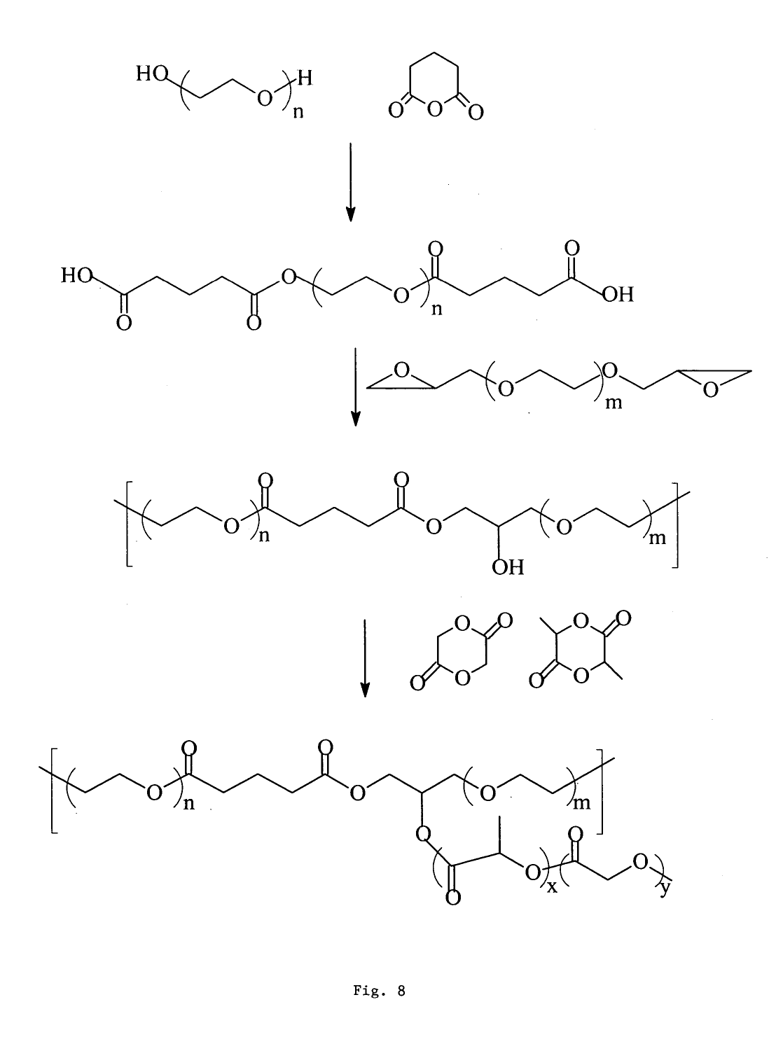

A 5% (by weight) solution of NiPAAm/AAc in PBS, containing also a small amount (>0.01%) of the blue dye was prepared and cooled in an ice bath. Small aliquots (0.2-0.3 ml) of the cold polymer solution were injected into the freshly isolated canine lymph nodes. After the injection, lymph nodes were kept at 37° C. for 10-15 minutes permitting the thermal gelation of the injected copolymer solution. The injected lymph nodes were then cut open with a razor blade to evaluate the extent of tissue perfusion. As shown in FIG. 7A, the dye perfusion within the lymph node 300 was limited to the extent of perfusion of the gelled copolymer solution 302, and was clearly visible.

As a control, dye solution in PBS only was injected into another lymph node 304 without mixing the dye into the gelling solution. Dye 306 was not contained locally within the lymph node but diffused throughout and beyond the lymph node as illustrated in FIG. 7B. Injection of the dye solution alone resulted in no dye localization within the lymph node 304.

### EXAMPLE 11

An experiment was conducted to demonstrate containment of a radioisotope in a gel. A non-radioactive stable isotope proxy was used. Specifically, in vitro experiments were performed to evaluate the ability of the poly(NiPAAm-co-AAc) copolymer gel to entrap stable isotope forms of barium and yttrium salts within the gel matrix at 37° C.

The poly(NiPAAm-co-AAc) copolymer containing 2 mole % of acrylic acid was used for the experiments in this example. This copolymer exhibited the gelling transition at about 37° C. The following salts of barium and yttrium were tested: yttrium chloride, barium chloride and barium sulfate.

Yttrium Chloride (YCl₃)

Polymer solution (1) was prepared by dissolving 5.0 g of poly(NiPAAm-co-AAc) copolymer in 95.0 g of phosphate buffered saline (PBS). YCl₃ colloidal suspension was prepared by mixing 0.11 g of YCl₃ with 10 ml of PBS. An amount of 0.1 ml of the colloidal suspension was added to 0.9 ml of polymer solution (1) and mixed thoroughly. The mixed polymer solution was then placed in a test tube and incubated at 37° C. for 20 minutes. Formation of an opaque gel was observed. The amount of YCl₃ in the gel was 1100 μg.

After 20 minutes of incubation, 10 ml of PBS prewarmed to 37° C. was added to the test tube on the top of the gel. The gel layer stayed intact for the time of the experiment, and the added PBS did not mix with the gel layer.

The concentrations of YCl₃ in the PBS were tested after 33 minutes and 24 hours by ICP/AES Analysis. Detected concentrations were 0.12 μg/ml after 30 minutes and 0.03 μg/ml after 24 hr. These correspond to 0.12% and 0.03% of total yttrium present in the gel. In conclusion, the poly(NiPAAm-co-AAc) copolymer gel localized YCl₃ very efficiently.

Barium Chloride (BaCl₂)

To prepare a BaCl₃ stock solution, 0.497 g of BaCl₂ was mixed with 5 ml PBS. Next, 0.1 ml of the stock solution was added to 0.9 ml of polymer solution (1) and mixed thoroughly. The mixed polymer solution mixture was then placed in a test tube and incubated at 37° C. for 20 minutes. Formation of an opaque gel was observed. The amount of BaCl₂ in the gel was 9036 μg.

After 20 minutes of incubation, a 10 ml of PBS prewarmed to 37° C. was added to the test tube on the top of the gel. The gel layer stayed intact for the time of the experiment, and the added PBS did not mixed with the gel layer.

The concentrations of BaCl₂ in the PBS were tested after 33 minutes and 24 hours by ICP/AES Analysis. Detected concentrations were 30.95 μg/ml after 30 minutes and 17.73 μg/ml after 24 hr. These correspond to 3.76% and 2.16% of the total barium present in the gel. In conclusion, the poly(NiPAAm-co-AAc) copolymer gel localized BaCl₂ less efficiently than YCl₃. The difference was due mainly to a higher water solubility of the BaCl₂ salt.

Barium Sulfate (BaSO₄)

BaSO₄ exhibits very low solubility in water. In order to prepare a BaSO₄/polymer suspension, 10.1 mg of BaSO₄ was added directly to 0.99 ml of polymer solution (1) and mixed thoroughly. The mixed polymer solution was then placed in a test tube and incubated at 37° C. for 20 minutes. Formation of an opaque gel was observed. The amount of BaSO₄ in the gel was 10,100 μg.

After 20 minutes of incubation, a 10 ml of PBS prewarmed to 37° C. was added to the test tube on the top of the gel. The gel layer stayed intact for the time of the experiment, and the added PBS did not mixed with the gel layer.

The concentrations of BaSO₄ in the PBS were tested after 33 minutes and 24 hours by ICP/AES Analysis. Detected concentrations were 0.34 μg/ml after 30 minutes and 0.28 μg/ml after 24 hours. These correspond to, respectively, 0.04% and 0.03% of the total barium present in the gel. In conclusion, the poly(NiPAAm-co-AAc) copolymer gel localized BaSO₄ equally efficiently as YCl₃.

### EXAMPLE 12

This example demonstrates the in vivo containment of a radioisotope carrier in a cancerous tumor.

A preliminary study was conducted in mice to demonstrate administering therapeutic levels of polymer composite (gel) containing yttrium-90 to tumors growing in mice. The purpose of this study was to show that (1) Y-90 was contained within the polymer composite at the site of the injection, and that (2) cancer cells in the tumors were killed by the intense beta-particle radiation from the Y-90 in the polymer. It is well known that beta particle radiation is useful for treating cancer cells. However, the melanoma cell line is a radiation-resistant cancer requiring very high radiation doses for effective therapy. Very high radiation doses (greater than 1000 Gy, or 100,000 rads) would be needed for complete cell killing in melanoma. Therefore, the main objective of this study was to demonstrate the feasibility of safely delivering very high radiation absorbed doses to live mice.

Materials and Methods:

Animals Twelve normal, six-to-eight-week old C57BL/6 female mice were obtained from Charles River Laboratories, were acclimatized on standard shredded pine-chip bedding in plastic laboratory cages, and were provided standard rodent chow and water ad libitum.

All procedures and experiments were approved in advance by the Institutional Animal Care Committee.

Inoculations

Each of the 12 mice was inoculated with melanoma cells 12 days prior to the scheduled therapy injection. The cell line used was B16-F1 mouse melanoma from the 331 building stock. Approximately 50,000 viable tumor cells in 0.05 mL of cell culture medium (a concentration of about 1 million cells per mL) were administered subcutaneously into the inner anterior thigh/abdomen area.

Materials

Yttrium-90 chloride (90-YCl₃) was prepared as a solution in 0.05M HCl. Colloidal Y-90 was formed upon mixing with polymer solution in phosphate buffered saline (PBS) (this was not prepared hydrothermally). Approximately 2.6 mCi Y-90 were placed in each of six heavy shielded (beveled glass) vials (0.1 mL Kimax) stored in shielded aluminum overpack.

The polymer composite used was a thermally reversible copolymer gel composed of poly(N-isopropylacrylamide-co-acrylic acid)[NA2 polymer], as a 5% (by weight) solution in PBS. This thermally sensitive polymer is liquid at room temperature (about 22° C.). It solidifies at body temperature (about 37° C.). Approximately 0.1 mL of polymer was drawn by syringe and place into the Kimax vial containing the 2.6 mCi Y-90, and mixed prior to injection into a mouse tumor.

Approximately 0.1 mL Dexamethasone sodium phosphate U.S.P. (Dex), 3 mg/mL (Steris Laboratories, Inc., Phoenix) was administered to some of the tumors by needle injection. The purpose of the Dex was to reduce the intratumoral pressure. Fifteen minutes elapsed between injection of Dex and injection of Y-90 polymer composite.

Rubber-shielded syringes (0.5 to 1.0 cc) fitted with micro-fine needles (27 gauge) were used to administer the radioisotope composite solution to the mouse tumors.

Radioisotope Polymer Composite Injection

Ten mice were anesthetized, one at a time, with methoxyflurane (Pitman-Moore, N.J.). The mice were kept intermittently under an infra-red heating lamp to keep their body temperature at or above 37° C.

Each of these ten mice was injected with dexamethasone, NA2 polymer, or Y-90 solutions according to the treatment regimen summarized in Table 2.

Mice were sacrificed on day two after the therapy injection by CO₂ asphyxiation, and their tumor, liver, kidneys and spleen were excised and preserved for further examination and radiological counting for Y-90. The amount of Y-90 in the excised tissues (liver, kidney and spleen) was determined using a planar beta counter. The histopathology of the tumors was also examined.

TABLE 3
Injection regimes and survival.

| Mouse | Injection regime (total injection | |
|---|---|---|
| number | volume 100 μl) | Comments |
| 1 | Dexamethasone + polymer + Y-90 | Alive on day 2, some skin |
| | | burning observed on |
| | | periphery of tumor |
| 2 | Dexamethasone + polymer + Y-90 | Alive on day 2 |
| 3 | Dexamethasone + polymer + Y-90 | Alive on day 2 |
| 4 | Polymer + Y-90 | Alive on day 2 |
| 5 | Polymer + Y-90 | Died on day 1, 2 large |
| | | tumors |
| 6 | Polymer + Y-90 | Alive on day 2 |
| 7 | Dexamethasone + polymer | Died on day 2 |
| 8 | Dexamethasone + polymer | Died on day 1 |
| 9 | Polymer | Alive on day 2 |
| 10 | Polymer | Died on day 1 |
| 11 | No treatment | Died on day 2 |
| 12 | No treatment | Died on day 2 |

TABLE 4
Weights of the excised tumors and organs on day 2 (necropsy).

| Mouse | | | | |
|---|---|---|---|---|
| Number | Tumor [g] | Liver [g] | Kidney [g] | Spleen [g] |
| 1 | 3.4 | 0.8 | 0.32 | 0.03 |
| 2 | 1.6 | 0.64 | 0.40 | — |
| 3 | 3.5 | 0.7 | 0.25 | 0.04 |
| 4 | 1.78 | 0.89 | 0.32 | 0.05 |
| 6 | 1.23 | 0.74 | 0.38 | 0.08 |
| 7 | 1.2 | 0.98 | 0.32 | 0.07 |
| 9 | 2.4 | 0.60 | 0.28 | 0.07 |

TABLE 5
Isotope containment within the tumors.

| | | Activity per sample on day 2 after injection | | | | | |
|---|---|---|---|---|---|---|---|
| | Total Y-90 | (microcuries, decay-corrected to time of | | | | | |
| | activity | injection), and percent of administered activity | | | | | |
| Mouse | administered | Kidney | | Liver | | Spleen | |
| number | (microcuries) | μCi | % | μCi | % | μCi | % |
| 1 | 2600 | 17.1 | 0.658 | 8.29 | 0.319 | 0.44 | 0.017 |
| 2 | 2600 | 40.0 | 1.538 | 16.9 | 0.650 | — | — |
| 3 | 2600 | 6.76 | 0.260 | 3.40 | 0.131 | 0.092 | 0.034 |
| 4 | 2600 | 18.8 | 0.723 | 19.4 | 0.746 | 0.71 | 0.027 |
| 5* | 2600 | — | — | — | — | — | — |
| 6 | 2600 | 9.91 | 0.381 | 3.33 | 0.128 | 0.11 | 0.004 |

*Mouse 5 died before necropsy

Pathology Report

Formalin fixed specimens from 7 mice were submitted in individual vials. The specimens were trimmed, inserted in plastic cassettes, and submitted to Our Lady of Lourdes Hospital in Pasco, Wash., for the preparation of paraffin sections stained with hematoxylin and eosin.

The following information was provided: the mice had been injected subcutaneously with a melanoma cell line; the resulting tumors from 5 of the 7 mice (specimens labeled 1,2,3,5, and 6) were injected with a soluble matrix of yttrium-90; the mice were killed 2 days after Y-90 injection; a high radiation dose from the Y-90 was delivered to the tumors; the specimens were stored for several weeks to allow for the decay of the Y-90; tumors from mice #1,2,3, and 7 were also injected with a corticosteroid; mice #7 and 9 were not injected with Y-90.

The purpose of the histopathologic examination was to determine whether the Y-90 had an effect on the tumors, and how that effect could be altered with corticosteroids.

Pathology Discussion

The tumors produced by the injected cell cultures resembled malignant melanomas. The necrosis observed was compatible with tumors of a high level of malignancy and also with the effect of high-dose irradiation and cell death. There were no clear difference in the necrosis between the Y-90 injected tumors and the tumors not injected with Y-90. It should be noted that the measurements provided were for only one sample from each specimen and are not intended to imply quantitative differences between the specimens.

The short (2-day) time period between therapy injection of Y-90 polymer composite and sacrifice would not have been sufficient for manifestation of extensive cell-killing. The tumors were well-advanced and necrotic, which made it difficult to isolate the radiotoxic effects of high-dose irradiation. In general, the mice treated with Y-90 appeared to be more healthy and active, even with large tumor burdens on day 2 after the injection, just before necropsy, than did the mice that were not treated with Y-90. The notable exception was mouse No. 5 with two large tumors. Mouse No. 5 died of causes apparently related to melanoma tumor burden on day 1.

Radiation Absorbed Dose Estimates The Y-90 equilibrium dose constant is 1.99 g-rad/μCi-hr. The cumulated hours in the tumor for two days is 21.1 hrs (the integral of the time-activity curve for 100% retention in the tumor). Therefore, the radiation absorbed dose to a tumor of 3.5 g would be 1.99×2600×21.1 /3.5=31,200 rads (312 Gy). For a 1.7 g tumor, the dose would be 64,200 rads (642 Gy).

Actual doses to tumors would be less than this amount, because some of the activity was excreted, and a fraction translocated to normal tissues.

Summary and Conclusions

This experiment demonstrated the administration of a radioisotope polymer composite to solid tumors in animals. The experiment clearly demonstrated the ability of the gelling polymers, such as NA2 polymer, to contain Y-90 radioisotope mostly at the injection site. The tumors remained very radioactive after administration, showing that most of the injected activity was retained at the injection site. Normal-tissue uptake was minimal (a few percent or less). Histopathology did not show a significant melanoma cell death from radiation because of the short time between injection and necropsy; however, the efficacy of treatment may have been obscured by the short time between injection and necropsy, and the advanced stage of the tumor development (extensive coagulative necrosis).

### EXAMPLE 13

An experiment was conducted to demonstrate placement of Yttrium-90/technetium-99m-polymer composite into prostate tissue of two young, healthy beagle dogs.

Y-90/Tc-99m-polymer Composite

Yttrium-90 was prepared as a solution of YCl₃ in 0.05 N HCl. Colloidal Y-90 was formed upon mixing with polymer solution in phosphate buffered saline (this was not hydrothermally synthesized). The total amount administered to each dog prostate was approximately 1.1 mCi in a volume of 1.5 to 2.0 mL. Technetium-99m sulfur colloid (Mallincrodt Medical) (600 μCi, or 22 MBq, 100 μL by volume) was added to and mixed with the Y-90-polymer solution immediately prior to injection to facilitate in vivo gamma-camera imaging of the injected material.

Reversibly gelling copolymers of N-isopropylacrylamide and acrylic acid, poly(NiPAAm-co-acrylic acid), were tested as delivery vehicles for the Y-90 and Tc-99m radioisotopes. Two copolymers with different gelling temperatures were tested: NA-2 Sept 97, with gelling temperature at 37° C. and NA-1.8 Feb98/2 that gels at 36° C. NA-2 Sept 97 was synthesized using slightly more acrylic cid than NA-1.8 Feb 98/2, which accounts for its higher selling temperature.

Polymer solutions were prepared having 5% (by weight) of either poly(NiPAAm-co-acrylic acid) copolymer dissolved in PBS. A 50 μL solution of YCl₃ (stable yttrium) suspension was added to this solution to obtain a diluted YCl₃ suspension with total stable yttrium concentration of 55 μg/mL. The suspension was steam-sterilized in an autoclave for 15 minutes. Directly before injection, an appropriate amount of this suspension was added to the vial containing the Y-90 radioisotope and mixed thoroughly.

Animals

Two beagle dogs were anesthetized for this study. Anesthesia given was injected Pentothal (15 mg/kg), with 0.625 Acepromazine and the same amount of Atropine plus Isoflurane gas and oxygen. The dogs' rectal temperatures were measured before and after the injections. The dogs were placed in a supine position on the operating table, and were immobilized. The hind legs were tied upward to facilitate the polymer injections. The ultrasound probe was placed in the rectum. The parallel grid template was affixed to the ultrasound probe.

Injection Procedure The Y-90/Tc-99m-polymer composite was injected using two to four needles inserted through a parallel grid template.

Gamma Imaging

Portable gamma camera was used to image the localization of technetium-99m gamma rays from the polymer composite.

Dog 1 Procedure and Results

The initial rectal temperature was 38.0° C., and the final temperature was 36.3° C.

The surgeons had difficulty identifying/defining the position of the prostate due to a large pubic arch and small gland size (2.5 cm wide, 1.5 cm int.-post.), volume approx. 7 cm³.

The amount and polymer batch injected was 1.6 mL of NA-2 Sept 97, having a gelling temperature 37° C.

The Y-90/Tc-99m-polymer composite was maintained on ice before injection. Two needles were used, and four squirts of about 0.2 mL each were administered through each needle.

Gamma-camera imaging was performed immediately after the polymer composite was administered to the prostate gland. The images showed liver uptake (approximately 20% within 10 minutes after the injection). A possible cause for early liver uptake may have been a direct injection into the vasculature and trauma to the prostate after multiple needle puncture. The polymer may not have had an opportunity to gel completely because the dog body temperature was slightly below the polymer gelation point (36.3° C. vs. 37° C.).

Dog 2 Procedure and Results

The initial rectal temperature was not measured. The final rectal temperature was 36.6° C. The prostate of Dog 2 was easier to locate. There was no pubic arch problem. The prostate gland was slightly larger, with an estimated volume of 10 cm³ (4×3 cm).

The amount and polymer batch administered were 2.0 mL of NA-1.8 Feb98/2, having a gelling temperature 36° C. The Y-90-polymer composite was maintained at room temperature prior to injection into Dog 2. Four needles were used to administer the polymer composite. Injections consisted of 0.5 mL (500 μL) per needle as three squirts per needle of about 0.17 mL per squirt. Each squirt was given after withdrawing the needle approximately 1 cm.

Gamma-camera imaging was conducted immediately after the injections, acquiring approximately 200,000 counts. The images showed no specific liver uptake within 10 minutes after the injection.

It appears that the polymer did gel completely in Dog 2 because the dog body temperature was slightly above the polymer gelation point (36.6° C. vs. 36° C.).

Post Injection Procedures for Dog 1 and 2

Animals were sacrificed on day 9 after the injection of the Y-90/Tc-99m-polymer composites. Samples of the prostate, liver, lung, heart, kidney, and spleen tissues were collected and the activity of Y-90 in the tissues was determined in pCi/gm per unit wet weight. The results are summarized in Table 6. As shown, after nine days in vivo, the concentrations of Y-90 in the prostate of Dog 2 were significantly higher than concentrations of Y-90 in other tissues of the animal. Tissue counts for Dog 1 demonstrate no specific localization of Y-90 in the prostate tissue. These results are consistent with gamma imaging results showing that the surgeons completely missed the target tissue (prostate) during their injection procedure. There was also significant liver uptake of Tc-99m in Dog 1 and no liver uptake in Dog 2. The results are also consistent with the fact that polymer used for Dog 1 had a higher gelling transition temperature (37° C.) and did not gel during the injection because Dog 1 body temperature was below 37° C. (36.3° C.). Therefore radioisotope localization did not occur in Dog 1. However, localization did occur in Dog 2.

TABLE 6
The Y-90 activities measured in tissue samples
collected on day 9 post injection.

| | Wet Weight (g) | Y-90 Activity (pCi/gm) |
|---|---|---|
| Dog 1 | | |
| Heart | 0.6475 | 11.4 |
| Kidney | 0.4712 | 211 |
| Liver | 0.6510 | 214 |
| Lung | 0.3587 | 342 |
| Spleen* | 0.0704 | 51.5 |
| Prostate | 1.2450 | 37.7 |
| Dog 2 | | |
| Heart | 2.1700 | 4.44 |
| Kidney | 0.7028 | 112 |
| Liver | 1.1765 | 104 |
| Lung | 1.6691 | 362 |
| Spleen | 0.6935 | 50.4 |
| Prostate | 1.7588 | 13650 |

Conclusions

The results of Y-90 counts and gamma imaging are consistent with the fact that gelling temperatures of the injected polymers were different and that polymer used for the Dog 1 procedure did not completely gel (the Dog 1 body temperature was slightly below the polymer gelation point: 36.3° C. vs. 37° C.). Hence, the results presented clearly illustrate the importance of gelling polymer in localization of the injected radioisotopes.

This experiment showed localization of radioisotope polymer composite in the target tissue (prostate) of Dog 2 with minimal radionuclide activity in other non-target tissues at 9 days post injection. Subsequent autoradiography of Dog 2 prostate showed uniform distribution of the Y-90 polymer composite throughout the prostate gland.

For the therapeutic agent carrier compositions disclosed above, the therapeutic agent may comprise, in some embodiments, from about 0.1% to about 50% percent by weight of the total composition (for the polymer carriers, the total composition weight does not include aqueous solvent, which may be used to form a gelling polymer solution and typically is 70% to about 99% (by weight) of the solution). The compositions are formed by simply mixing the therapeutic agent with the therapeutic agent carrier, such as by stirring, agitation, or just by adding one component into the other. For example, in one embodiment a ⁹⁰YPO₄ colloid is mixed with fibrin to form a therapeutic agent carrier composition.

When using a radioactive therapeutic agent as the therapeutic agent, the percentage of the therapeutic agent used by weight is not a controlling factor for the administered dosage. For example, though the carrier can accommodate at least as much as 50% by weight of therapeutic agent, it is simple to just vary the radioactivity of the radioactive therapeutic agent to deliver the desired dose of radiation rather than varying the amount of therapeutic agent used. For example, if a high dose of radiation is desired, a low percentage by weight of 90-yttrium colloid can be used, but such a colloid would comprise particles having an increased amount of 90-yttrium relative to the amount of 90-yttrium used in a lower dosage composition.

While the examples described above disclose specific non-limiting embodiments of the therapeutic agent carrier compositions disclosed herein, as well as methods of making and using such compositions, it will be apparent to those skilled in the art that many changes and modifications may be made without departing from the scope of the appended claims. The appended claims are therefore intended to cover all such changes and modifications as fall within their true spirit and scope.

We claim:

1. A composition comprising:
a therapeutic agent comprising hydrothermally synthesized 90-yttrium phosphate particles; and
a therapeutic agent carrier comprising fibrin, wherein the therapeutic agent is mixed with the therapeutic agent carrier.

2. The composition of claim 1, wherein the 90-yttrium phosphate particles have a mean diameter of from about 0.3 to about 3 μm.

3. The composition of claim 1, wherein the 90-yttrium phosphate particles have a mean diameter of from about 0.3 to about 0.8 μm.

4. The composition of claim 1, wherein the 90-yttrium phosphate particles have an average diameter of from about 0.3 to about 0.8 μm.

5. The composition of claim 1, wherein a substantial amount of the 90-yttrium phosphate particles are substantially spherical.

6. The composition of claim 2, wherein the therapeutic agent comprises a colloid, wherein the 90-yttrium phosphate particles comprise a disperse phase of the colloid.

7. The composition of claim 6, wherein the therapeutic agent carrier further comprises a stimulus sensitive gelling polymer.

8. The composition of claim 7, wherein the stimulus sensitive gelling polymer is a thermogelling biodegradable polymer comprising a polyethylene glycol block and a biodegradable polyester block, wherein said blocks are linked to form a polymer of a general structure satisfying the formula Aₙ(B), wherein n is equal to or greater than 2, A is selected from the group consisting of a polyethylene glycol block and a biodegradable polyester block, B is selected from the group consisting of a polyethylene glycol block and a biodegradable polyester block, and A is different than B.

9. The composition of claim 7, wherein the stimulus sensitive gelling polymer is a thermogelling biodegradable polymer carrier comprising a biocompatible polymer block and a biodegradable polypeptide block, wherein the biocompatible polymer and the polypeptide blocks are linked to form a polymer of a general structure satisfying the formula CₙDₘ, wherein n is equal to or greater than 1, m is equal to or greater than 1, C is a biodegradable polypeptide block, and D is a biocompatible soluble polymer having a chain length such that if D is not biodegradable, D may be eliminated through a glomeruli filtration system.

10. A composition comprising,
a therapeutic agent carrier comprising a thermogelling, biodegradable, polymer comprising a polyethylene glycol block and a biodegradable polyester block, wherein said blocks are linked to form a polymer of a general structure satisfying the formula Aₙ(B), wherein n is equal to or greater than 2, A is selected from the group consisting of a polyethylene glycol block and a biodegradable polyester block, B is selected from the group consisting of a polyethylene glycol block and a biodegradable polyester block, and A is different than B; and
a therapeutic agent comprising hydrothermally synthesized 90-yttrium phosphate particles, wherein the therapeutic agent is mixed with the therapeutic agent carrier.

11. The composition of claim 10, wherein n is between 3 and 10.

12. The composition of claim 11, wherein the polyethylene glycol has an average molecular weight of between 200 and 20,000.

13. The composition of claim 10, wherein the 90-yttrium phosphate particles have a mean diameter of from about 0.3 to about 3 μm.

14. The composition of claim 13, wherein the 90-yttrium phosphate particles have a mean diameter of from about 0.3 to about 0.8 μm.

15. The composition of claim 10, wherein the 90-yttrium phosphate particles have an average diameter of from about 0.3 to about 0.8 μm.

16. The composition of claim 14, wherein a substantial amount of the 90-yttrium phosphate particles are substantially spherical.

17. The composition of claim 14, wherein about 40% of the 90-yttrium phosphate particles are substantially spherical.

18. The composition of claim 13, wherein the therapeutic agent comprises a colloid, wherein the 90-yttrium phosphate particles comprise a disperse phase of the colloid.

19. A composition comprising:
a therapeutic agent carrier comprising a thermogelling, biodegradable, polymer carrier comprising a biocompatible polymer block and a biodegradable polypeptide block, wherein the biocompatible polymer and the polypeptide blocks are linked to form a polymer of a general structure satisfying the formula CₙDₘ, wherein n is equal to or greater than 1, m is equal to or greater than 1, C is a biodegradable polypeptide block, D is a biocompatible soluble polymer having a chain length such that if D is not biodegradable, D may be eliminated through a glomeruli filtration system; and
a therapeutic agent comprising hydrothermally synthesized 90-yttrium phosphate particles, wherein the therapeutic agent is mixed with the therapeutic agent carrier.

20. The composition of claim 19, wherein D comprises a polyethylene glycol block.

21. The composition of claim 20, wherein the polypeptide block has an average molecular weight of from about 300 to about 30,000.

22. The composition of claim 19, wherein the 90-yttrium phosphate particles have a mean diameter of from about 0.3 to about 3 μm.

23. The composition of claim 22, wherein the 90-yttrium phosphate particles have a mean diameter of from about 0.3 to about 0.8 μm.

24. The composition of claim 22, wherein a substantial amount of the 90-yttrium phosphate particles are substantially spherical.

25. The composition of claim 22, wherein the therapeutic agent comprises a colloid, wherein the 90-yttrium phosphate particles comprise a disperse phase of the colloid.

26. A method for making a therapeutic agent carrier comprising:
forming a therapeutic agent carrier comprising a stimulus-sensitive gelling polymer, fibrin, or combinations thereof; and
mixing a hydrothermally synthesized radioactive therapeutic agent with the therapeutic agent carrier.

27. The method of claim 26, wherein hydrothermally synthesizing the radioactive therapeutic agent comprises hydrothermally synthesizing a 90-yttrium phosphate colloid by reacting a yttrium source, EDTA, and a phosphate source.

28. The method of claim 27, wherein the 90-yttrium source, EDTA, and phosphate source are reacted in an acidic environment.

29. The method of claim 28, wherein the 90-yttrium source, EDTA, and phosphate source are reacted at a pH of from about 6 to about 6.5.

30. The method of claim 28, wherein the 90-yttrium source, EDTA, and phosphate source are reacted at a temperature of at least about 150° C.

31. The method of claim 30, wherein the 90-yttrium source, EDTA, and phosphate source are reacted for at least about 20 hours.

32. The method of claim 27, wherein the yttrium source comprises ⁹⁰YCl₃.

33. The method of claim 32, wherein the phosphate source comprises NaH₂PO₄.

34. The method of claim 27, wherein the 90-yttrium source, EDTA, and phosphate source are combined in a yttrium source:EDTA:phosphate source ratio of about 1:1:6.

35. A method of treating cancer in a subject comprising:
resecting a tumor from a subject, wherein resection of the tumor creates a tumor removal site; and
applying a therapeutic agent carrier composition to intact tissue at the tumor resection site in an amount effective to destroy vestigial cancerous cells, wherein the therapeutic agent carrier composition comprises a hydrothermally synthesized 90-yttrium phosphate colloid and a therapeutic agent carrier, wherein the therapeutic agent carrier is a) fibrin, b) a thermogelling, biodegradable polymer carrier comprising a polyethylene glycol block and a biodegradable polyester block, wherein said blocks are linked to form a polymer of a general structure satisfying the formula Aₙ(B), wherein n is equal to or greater than 2, and A is selected from the group consisting of a polyethylene glycol block and a biodegradable polyester block, B is selected from the group consisting of a polyethylene glycol block and a biodegradable polyester block, and A is different than B, c) a thermogelling, biodegradable polymer carrier comprising a biocompatible polymer block and a biodegradable polypeptide block, wherein the biocompatible polymer and the polypeptide blocks are linked to form a polymer of a general structure satisfying the formula CₙDₘ, wherein n is equal to or greater than 1, m is equal to or greater than 1, C is a biodegradable polypeptide block, D is a biocompatible soluble polymer having a chain length such that if D is not biodegradable, D may be eliminated through a glomeruli filtration system, or d) combinations thereof.
